SEQUENCE LISTING (35 sequences)

SEQ ID NO: 1 - DNA, 20 bp, Artificial Sequence
  MF1F Primer
agrgtttgatcmtggctcag

SEQ ID NO: 2 - DNA, 36 bp, Artificial Sequence
  MF1F-con Primer
agrrttygatcmtggytcagrnykaacgctggcggc

SEQ ID NO: 3 - DNA, 18 bp, Artificial Sequence
  MF1R Primer
gttactcrcccgtkcgcc

SEQ ID NO: 4 - DNA, 18 bp, Artificial Sequence
  MF1aR Primer
gttactcrcccgtncgcc

SEQ ID NO: 5 - DNA, 17 bp, Artificial Sequence
  MF1bR Primer
gttactcrcccgtkcgc

SEQ ID NO: 6 - DNA, 17 bp, Artificial Sequence
  MF1cR Primer
gttactcrcccgtncgc

SEQ ID NO: 7 - DNA, 17 bp, Artificial Sequence
  MF1dR Primer
ttactcrcccgtkcgcc

SEQ ID NO: 8 - DNA, 17 bp, Artificial Sequence
  MF1eR Primer
ttactcrcccgtncgcc

SEQ ID NO: 9 - DNA, 16 bp, Artificial Sequence
  MF1fR Primer
ttactcrcccgtkcgc

SEQ ID NO: 10 - DNA, 16 bp, Artificial Sequence
  MF1gR Primer
ttactcrcccgtncgc

SEQ ID NO: 11 - DNA, 18 bp, Artificial Sequence
  MF1R-con Primer
gttactyrcccntncrcy

SEQ ID NO: 12 - DNA, 18 bp, Artificial Sequence
  MF2F Primer
tcctacgggaggcagcag

SEQ ID NO: 13 - DNA, 28 bp, Artificial Sequence
  MF2-con Primer
actcctacgggaggcagcagtngrgaat

SEQ ID NO: 14 - DNA, 18 bp, Artificial Sequence
  MF2R Primer
tgctggcacgkagttagc

SEQ ID NO: 15 - DNA, 34 bp, Artificial Sequence
  MF2R-con Primer
ygtatyaccgcrrctgctggcacrnagttrgycg

SEQ ID NO: 16 - DNA, 16 bp, Artificial Sequence
  MF3aF Primer
agtagtggcgagcgaa

SEQ ID NO: 17 - DNA, 25 bp, Artificial Sequence
  MF23 Primer
gatwycsnnagtagtggcgagcgaa

SEQ ID NO: 18 - DNA, 25 bp, Artificial Sequence
  MF3F-con Primer
gattnnnnnagtagtggcgagcgaa

SEQ ID NO: 19 - DNA, 23 bp, Artificial Sequence
  MF3R Primer
cttttcgcckttccttcacggta

SEQ ID NO: 20 - DNA, 44 bp, Artificial Sequence
  MF3R-con Primer
rgtncttttcrccnttccrtcacrgtactnntncnctatcgntn

SEQ ID NO: 21 - DNA, 22 bp, Artificial Sequence
  MF4F Primer
ttstngttaaggaactmkgcaa

SEQ ID NO: 22 - DNA, 17 bp, Artificial Sequence
  MF4aF Primer
gttaaggaactmkgcaa

SEQ ID NO: 23 - DNA, 23 bp, Artificial Sequence
  MF4F-con Primer
aaynnngnnaaggaactmkgcaa

SEQ ID NO: 24 - DNA, 17 bp, Artificial Sequence
  MF2R Primer
gcayygggcaggngtca

SEQ ID NO: 25 - DNA, 28 bp, Artificial Sequence
  MF2R-con Primer
ttaaccttnnrgcayygggcaggygtca

SEQ ID NO: 26 - DNA, 22 bp, Artificial Sequence
  MF2R Primer
caaggaatttcgctaccttagg

SEQ ID NO: 27 - DNA, 21 bp, Artificial Sequence
  MF2aR Primer
caaggaatttcgctaccttag

SEQ ID NO: 28 - DNA, 85 bp, Artificial Sequence
  MF2R-con Primer
ccwttcrwgcrggwcggwayttacccgacaaggaatttcgctaccttaggaysgttatagttacsrccgccrttyacyngggctt

SEQ ID NO: 29 - DNA, 2904 bp, Escherichia coli
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

SEQ ID NO: 30 - DNA, 1542 bp, Escherichia coli
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgagttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 31 - DNA, 18 bp, Artificial Sequence
  ME20R Primer
acaacccgaaggccttct

SEQ ID NO: 32 - DNA, 18 bp, Artificial Sequence
  ME21R Primer
cgtcaatgagcaaaggta

SEQ ID NO: 33 - DNA, 18 bp, Artificial Sequence
  ME22R Primer
cgccgctgctggcacgga

SEQ ID NO: 34 - DNA, 18 bp, Artificial Sequence
  ME23R Primer
tgcgctttacgcccagta

SEQ ID NO: 35 - DNA, 18 bp, Artificial Sequence
  ME24R Primer
gctacacctggaattcta

(19) United States
(12) Patent Application Publication
(10) Pub. No.: US 2009/0263809 A1
(43) Pub. Date: Oct. 22, 2009

(54) Methods for Identification of Bioagents

(75) Inventors: Anthony M. Roberton, Auckland (NZ); David J. Saul, Auckland (NZ)

Correspondence Address: MORRISON & FOERSTER LLP, 425 MARKET STREET, SAN FRANCISCO, CA, 94105-2482, US

(73) Assignee: ZyGem Corporation Limited, Hamilton (NZ)

(21) Appl. No.: 12/408,645

(22) Filed: Mar. 20, 2009

Related U.S. Application Data
(60) Provisional application No. 61/038,389, filed on Mar. 20, 2008.

(51) Int. Cl. C12Q 1/68; B01D 57/02; G01N 27/26

(52) U.S. Cl. 435/6; 204/450

(57) ABSTRACT

Methods for detecting and identifying known and unknown bioagents, particularly bacteria, by nucleic acid amplification and amplicon size determination are provided. Nucleic acid amplification utilises multiple primers which hybridize to conserved bioagent ribosomal sequence regions and which bracket variable bioagent ribosomal sequence regions to produce multiple amplicons that uniquely identify the bioagent.

### RELATED APPLICATION

This application claims benefit under 35 USC 119(e) to U.S. Provisional Patent Application No. 61/038,389, filed Mar. 20, 2008, the contents of which is incorporated herein by reference in its entirety.

### FIELD

This application relates to methods for accurately and robustly identifying bioagents, particularly microorganisms and microorganisms in mixed populations, using nucleic acid methods.

### BACKGROUND

Methods to accurately and robustly identify bioagents, including microorganisms such as bacteria and viruses, are desirable for a variety of medical, industrial, environmental, quality control, security, and research reasons. Ideally, such methods are also rapid and yield accurate results quickly, in order for example to quickly allow a clinician to diagnose an infection or to minimise opportunities for exposure to dangerous organisms. In an industrial setting, an example may be to rapidly detect a change in a microbial population structure to prevent failure of a process, for example in waste water treatment. In environmental monitoring, rapid analysis of the microbial components of an ecosystem can provide invaluable data for assessing the nature, source and extent of deleterious changes.

Traditional microbiological methods typically identify microorganisms through direct examination and culture of specimens. For example, the standard method for bacterial identification used routinely in hospitals and laboratories is by culture of samples on selective or non-selective (rich nutrient supplied) agar plates, followed by morphological analysis of colonies or microbiological analysis using stains such as the Gram stain or using diagnostic biochemical tests.

However, a number of problems arise from these practices. These include: (i) the time taken to obtain a presumptive identification, which ranges from 12 hours to several weeks; (ii) unculturable, or senescent or dead bacteria are not identified; (iii) mixed cultures are frequently difficult to resolve, because one strain can swamp others—particularly if the important bacterium is slow growing, or if the time between the specimen being taken and a portion being plated is lengthy; (v) samples may need to be transported to a laboratory in suitable transport medium; and (vi) conditions for growth need to be correctly predetermined—for example, (a) a suitable medium must be selected, (b) anaerobic or microaerophilic bacteria will require a suitable gas atmosphere, (c) antibiotics may be needed to prevent dominant bacteria or yeast or fungal growth and (d) preliminary enrichment cultures may be required. These difficulties frequently lead to samples that are reported as “no growth observed”, or “mixed infection”, or only the faster growing bacterium in a mixed culture is reported.

A number of molecular methods for the identification of bioagents exist. For example, specific antibodies can be used to recognize specific bacteria, but cannot be used to identify unknown bacteria. Other molecular methods are reliant on specific hybridization, where small oligonucleotides or polynucleotides, tagged with a probe, can be used which hybridize specifically to a region of DNA in a selected bacterium.

Other molecular methods include PCR-based detection of a specific species or a phylogenetic group of species using primers that are uniquely suited to the bacterium being looked for. PCR methods are fast, but if the initial presumptive identification is incorrect, the selected PCR primers will fail to detect the bioagent(s) present.

A more generic strategy is to use primer sets that amplify from conserved regions of an organism's genome that are proximal to variable regions. The amplified fragment can then be subjected to restriction fragment polymorphism analysis or DNA sequencing to determine the identity of the organism. Although effective, this method is not as fast as direct PCR, and it is unsuitable for mixed populations of organisms or for discriminating related organisms.

If a pure culture of an organism is available, the Small Subunit rRNA gene can be amplified using generic primers, and the amplified DNA then sequenced. A BLAST search quickly identifies the unknown organism or places the sequence in a phylogenetic context among related species. Although this method is in principle the most reliable method of determining what organism is present, the procedure can take two to three days, and requires the availability of a pure culture. If only a mixed culture is available or if the bacterium cannot be grown, then a clone library of the amplified DNA must be generated, and then the DNA sequence of each type of insert determined. This process can take 2-3 weeks and is cost prohibitive.

A typical method that has been successfully used for this purpose is Amplified ribosomal DNA restriction analysis (ARDRA). This is a technique that accurately determines the number of different microbial types in a mixed culture, and obtaining the sequence of each clone type provides identification. The technique gives useful results, but is laboursome, costly and time-consuming. It will take a dedicated technician about 2-3 weeks to process 2 samples, and so the cost is usually prohibitive in all but research applications.

A simpler method is PCR amplification of the Internal Transcribed Sequence (ITS) of the ribosomal genes and to measure the size polymorphism of the amplicons between species. The method, sometimes known as rRNA Intergenetic Spacer Analysis (RISA) or Automated rRNA Intergenetic Spacer Analysis (ARISA) is useful for monitoring temporal changes in mixed microbial populations, but is limited in its ability to provide a definitive identification of the organisms present unless individual amplicons are isolated and their sequences determined (Fischer, M. M., and E. Triplett 1999. “Automated approach for ribosomal intergenic spacer analysis of microbial diversity and its application to freshwater bacterial communities”. Appl. Environ. Microbiol. 65:4630-4636).

DNA-based technologies have proven to be extremely useful for many diagnostic needs. For example, kits for the detection of fastidious organisms based on the use of hybridization probes or DNA amplification for the direct detection of pathogens from clinical specimens are commercially available (Tenover F. C., and E. R. Unger. 1993. “Nucleic Acid Probes for Detection and Identification of Infectious Agents”, pp. 3-25. In Persing, D. H., T. F. Smith, F. C. Tenover, and T. J. White (ed.) Diagnostic Molecular Microbiology: Principles and Applications.

U.S. Pat. No. 7,108,974, U.S. Pat. No. 7,226,739, and U.S. Pat. No. 7,255,992 describe methods for rapid detection and identification of bioagents using a combination of PCR and mass spectrometry. It is not clear that the methods described therein are sufficiently accurate to resolve complex mixtures of microbes. Moreover, the requirement for a mass spectrometer to analyse the results of the PCR renders these methods less likely to be adopted for some applications. For example because of the cost of re-equipping diagnostic laboratories. In addition, the 1-2% variation in DNA sequences observed commonly within species between the type strain and biological isolates makes mass accuracy problematic.

There is clearly a need for an accurate, rapid, and accessible method to identify bioagents, particularly microorganisms, encountered in a wide range of situations or in mixed populations. Ideally, such a method should use equipment and expertise easily accessed diagnostic laboratories.

Methods using nucleic acid techniques are provided herein.

### SUMMARY

In a first aspect, an embodiment provides a method for the identification of bacteria present in a sample wherein the method includes, but is not limited to:
contacting nucleic acid present in said sample with:
   a first pair of primers designed to produce a first amplicon from a 16S or 23S rRNA gene under amplification conditions;
   a second pair of primers designed to produce a second amplicon from a 16S or 23S rRNA gene under amplification conditions;
   a third pair of primers designed to produce a third amplicon from a 16S or 23S rRNA gene under amplification conditions; and
amplifying said nucleic acid with said first, second and third pair of primers;
determining the length of the amplicons produced; and
identifying the bacteria on the basis of the length of the amplicons.

In one embodiment, the method further includes contacting the nucleic acid present in said sample with a fourth pair of primers designed to produce a fourth amplicon from a 16S rRNA gene or a 23S rRNA gene under amplification conditions. In another embodiment, the method further includes contacting the nucleic acid present in said sample with a fifth pair of primers designed to produce a fifth amplicon from a 16S rRNA gene or a 23S rRNA gene under amplification conditions. In another embodiment, the method further includes contacting the nucleic acid present in said sample with a sixth pair of primers designed to produce a sixth amplicon from a 16S rRNA gene or a 23S rRNA gene under amplification conditions.

In a further embodiment, the second pair of primers is designed to produce a second amplicon from a 16S rRNA gene. In a further embodiment, the second pair of primers is designed to produce a second amplicon from a 23S rRNA gene.

In a further embodiment, the fourth pair of primers is designed to produce a fourth amplicon from a 16S rRNA gene. In a further embodiment, the fourth pair of primers is designed to produce a fourth amplicon from a 23S rRNA gene.

In a further embodiment, the fifth pair of primers is designed to produce a fifth amplicon from a 16S rRNA gene. In a further embodiment, the fifth pair of primers is designed to produce a fifth amplicon from a 23S rRNA gene.

In a further embodiment, the sixth pair of primers is designed to produce a sixth amplicon from a 16S rRNA gene. In a further embodiment, the sixth pair of primers is designed to produce a sixth amplicon from a 23S rRNA gene.

### Amplicon I

In a further embodiment, the first, second, fourth, fifth or sixth pair of primers is designed to selectively amplify a region of a 16S rRNA gene. Preferably the region of the 16S rRNA gene includes nucleotides 8 to 121 or nucleotides 8 to 120 or a region between nucleotides 7 and 122. Preferably the nucleotide position is taken from the nucleotide sequence of E. coli K12, strain MG 1566. The database accession number of the complete genome of E. coli K12 is NC_{—}000913 and the nucleotide sequence is incorporated herein by reference in its entirety. The nucleotide sequence of E. coli K12 16S rRNA is provided herein as SEQ ID No. 30.

In one embodiment, a pair of primers consists of a first primer that hybridizes to the 16S rRNA gene in the region of nucleotides 8 to 121, nucleotides 8 to 68 or nucleotides 8 to 43, and a second primer that hybridizes to the 16S rRNA gene in the region of nucleotides 8 to 121, nucleotides 98 to 121 or nucleotides 104 to 121. Preferably the pair of primers is the first, second, fourth, fifth or sixth pair of primers.

In a further embodiment, the pair of primers consists of a first primer of SEQ ID No. 1 or 2 and a second primer selected from SEQ ID NO.s 3 to 11. In another embodiment, the pair of primers consists of a first primer having 15 or more contiguous nucleotides of SEQ ID No. 2 and a second primer having 15 or more contiguous nucleotides of SEQ ID No. 11. See Tables 1 and 2 below for SEQ ID No.s 1 to 11.

### Amplicon II

In a further embodiment, the first, second, fourth, fifth or sixth pair of primers is designed to selectively amplify a region of a 16S rRNA gene having nucleotides 310 to 588, nucleotides 310 to 523, nucleotides 310 to 537, nucleotides 338 to 523, nucleotides 338 to 537, nucleotides 338 to 588, nucleotides 340 to 523, nucleotides 340 to 537 or nucleotides 340 to 588, or a region between nucleotides 309 and 589. Preferably the nucleotide position is taken from the nucleotide sequence of E. coli K12, strain MG1566.

In one embodiment, a pair of primers consists of a first primer that hybridizes to the 16S rRNA gene in the region of nucleotides 310 to 588, nucleotides 310 to 368 or nucleotides 338 to 365, and a second primer that hybridizes to the 16S rRNA gene in the region of nucleotides 310 to 588, nucleotides 504 to 588 or nucleotides 504 to 537. Preferably the pair of primers is the first, second, fourth, fifth or sixth pair of primers.

In a further embodiment, the pair of primers consists of a first primer of SEQ ID No. 12 or 13 and a second primer of SEQ ID No. 14 or 15. In another embodiment, the pair of primers consists of a first primer having 15 or more contiguous nucleotides of SEQ ID No. 13 and a second primer having 15 or more contiguous nucleotides of SEQ ID No. 15. See Tables 3 and 4 below for SEQ ID No.s 12 to 15.

### Amplicon III

In a further embodiment, the second, third fourth, fifth or sixth pair of primers is designed to selectively amplify a region of a 23S rRNA gene. Preferably the region of the 23S rRNA gene includes nucleotides 232 to 517, nucleotides 232 to 481, nucleotides 232 to 485, nucleotides 241 to 517, nucleotides 241 to 481, or nucleotides 241 to 485, or a region between nucleotides 231 and 518. Preferably the nucleotide position is taken from the nucleotide sequence of E. coli K12, strain MG1566. The nucleotide sequence of E. coli K12 23S rRNA is provided herein as SEQ ID No. 30.

In one embodiment, a pair of primers consists of a first primer that hybridizes to the 23S rRNA gene in the region of nucleotides 232 to 517 or nucleotides 232 to 256, and a second primer that hybridizes to the 23S rRNA gene in the region of nucleotides 232 to 517, nucleotides 442 to 517, nucleotides 442 to 485 or nucleotides 459 to 485. Preferably the pair of primers is the second, third, fourth, fifth or sixth pair of primers.

In a further embodiment, the pair of primers consists of a first primer of SEQ ID No. 16, 17 or 18 and a second primer of SEQ ID No. 19 or 20. In another embodiment, the pair of primers consists of a first primer comprising 15 or more contiguous nucleotides of SEQ ID No. 18 and a second primer having 15 or more contiguous nucleotides of SEQ ID No. 20. See Tables 5 and 6 below for SEQ ID No.s 16 to 20.

### Amplicon IV

In a further embodiment, the second, third fourth, fifth or sixth pair of primers is designed to selectively amplify a region of a 23S rRNA gene including nucleotides 1654 to 1971, nucleotides 1654 to 1945, nucleotides 1654 to 1854, nucleotides 1654 to 1843, 1656 to 1971, nucleotides 1656 to 1945, nucleotides 1656 to 1854, nucleotides 1656 to 1843, 1661 to 1971, nucleotides 1661 to 1945, nucleotides 1661 to 1854, nucleotides 1661 to 1843, or a region between nucleotides 1653 and 1972. Preferably the nucleotide position is taken from the nucleotide sequence of E. coli K12, strain MG 1566.

In one embodiment, a pair of primers consists of a first primer that hybridizes to the 23S rRNA gene in the region of nucleotides 1654 to 1854, nucleotides 1654 to 1704 or nucleotides 1654 to 1677, and a second primer that hybridizes to the 23S rRNA gene in the region of nucleotides 1654 to 1854, nucleotides 1818 to 1854 or nucleotides 1889 to 1971. Preferably the pair of primers is the second, third, fourth, fifth or sixth pair of primers.

In a further embodiment, the pair of primers consists of a first primer of SEQ ID No. 21, 22 or 23 and a second primer of SEQ ID No. 24, 25, 26, 27 or 28. See Tables 7 to 9 below for SEQ ID No.s 21 to 28.

In a further embodiment, the method further includes, but is not limited to:
contacting a nucleic acid of known identity with one or more primer pairs adapted to amplify said nucleic acid of known identity under amplification conditions to produce one or more calibration amplicons of a known length;
amplifying said nucleic acid with said one or more primer pairs; and
determining the length of the amplicons produced.

In a further embodiment, the said nucleic acid of known identity is 16S rRNA gene from E. coli K12, strain MG1566.

In a further embodiment, the bacteria present in the sample are one or more, two or more, three or more, four or more or five or more different genera, species or strains of bacteria. In one embodiment the bacteria are a mixed population and the sample is a sample for forensics, basic research, food monitoring, beverage monitoring, regulatory monitoring programs, veterinary monitoring or diagnostics, equine monitoring or diagnostics, agricultural monitoring and testing, horticultural monitoring and testing, livestock monitoring and diagnosis, clinical identifications and medical diagnosis (including diagnosis of infectious diseases and conditions, such as diagnosis in conjunction with drug resistance detection), environmental testing (e.g., detection and discrimination of pathogenic vs. non-pathogenic bacteria in water or other samples), germ warfare (allowing immediate identification of the bacteria and appropriate treatment), biosecurity monitoring or for any other application where the identification of bacteria to a useful level of discrimination is required.

In one embodiment, the bacteria are from one or more genera, for example, from Burkholderia, Escherichia, Lactobacillus, Ureoplasma, Streptococcus and Staphylococcus. In another embodiment, the bacteria are from one or more species, for example, B. cepecia, E. coli, L. gasseri, L. iners, L. jensenii, S. aureus, S. epidermidis, and St. pyogenes U. vaginalis.

In a further embodiment, the amplification using said first, second and third, and optionally fourth, or fourth and fifth, or fourth, fifth and sixth, pairs of primers is conducted simultaneously. Alternatively the amplification is conducted sequentially.

In a further embodiment, the amplification occurs in a single reaction mix.

In a further embodiment, the amplification is PCR amplification or isothermal amplification such as strand displacement amplification.

In a further embodiment, the length of the amplicons is determined by electrophoretic separation, such as capillary electrophoresis or gel electrophoresis.

In a further embodiment, at least one primer of each primer pair is labelled to allow for visual detection of amplicons. In one embodiment the labelled primer is labelled with a label selected from a specific binding ligand, an enzyme (such as glucose oxidase, peroxidases, uricase, and alkaline phosphatase), immunolabels, radiolabels, radioisotopes, electron-dense reagents, chromophores, chromogens, fluorogens or fluorophores, phosphorescent moieties, ferritin, nanodots or Q-dots, or any other type of label that can be used to differentiate amplicons. Preferably the label is a fluorophore. Examples of specific binding ligands include biotin, avidin and its homologues, antigens, or an antibody or antibody fragment (e.g. short chain variable fragments.

In a further embodiment, at least one primer of each primer pair is labelled with one of FAM, HEX, JOE, ROX, TMARA, TET.

In a further embodiment, each labelled primer has a different label to facilitate identification of amplicons.

In a further aspect, a nucleic acid having the sequence of one of SEQ ID NOs. 16 to 28 or a nucleic acid having at least 90% homology therewith and the ability to selectively hybridise to a 23S rRNA gene under stringent conditions is provided.

In a further aspect, a nucleic acid having the sequence of one of SEQ ID Nos. 1 to 15 or a nucleic acid having at least 90% homology therewith and the ability to selectively hybridise to a 16S rRNA gene under stringent conditions is provided.

In a further aspect, a primer pair designed to selectively amplify a region of 23S rRNA gene, the primer pair being selected from SEQ ID Nos. 16 to 28, is provided.

In a further aspect, a primer pair designed to selectively amplify a region of 16S rRNA gene, the primer pair being selected from SEQ ID Nos. 1 to 15, is provided.

In a further aspect, an embodiment provides a method for the identification of bacteria present in a sample, wherein the method includes at least the following steps:
providing the results of an analysis, the analysis including contacting nucleic acid present in said sample with:
   a first pair of primers designed to produce a first amplicon from a 16S rRNA gene under amplification conditions;
   a second pair of primers designed to produce a second amplicon from a 16S rRNA gene or a 23S rRNA gene under amplification conditions;
   a third pair of primers designed to produce a third amplicon from a 23S rRNA gene under amplification conditions; and
amplifying said nucleic acid with said first, second and third pair of primers; and determining the length of the amplicons produced; and
comparing the results to the length of the amplicons obtained or obtainable by performing the amplification on a plurality of known organisms, and
identifying the bacteria on the basis of results of the analysis.

In other aspects, an embodiment provides a system for performing one or more of the methods described, said system having, but not limited to, the following components:

computer processor means for receiving, processing and communicating data;

storage means for storing data including a reference genetic database of the results of genetic analysis of one or more bacteria; and

a computer program embedded within the computer processor which, once data consisting of or including the result of a genetic analysis for which data is included in the reference genetic database is received, processes said data in the context of said reference databases to determine, as an outcome, the identification of the bacteria, said outcome being communicable once known, preferably to a user having input said data.

Preferably, said system is accessible via the internet or by personal computer.

Preferably, the genetic analysis includes steps of the methods as described above.

More preferably, the reference genetic database includes the length of the amplicons associated with at least one bacterium.

In yet a further aspect, an embodiment provides a computer program suitable for use in a system as defined above including a computer usable medium having program code embodied in the medium for causing the computer program to process received data consisting of or including the result of at least one genetic analysis of one or more bacteria, in the context of a reference genetic database of the results of at least one genetic analysis.

In yet another aspect, an embodiment provides a kit for use according to a method described herein, the kit including three, four, five, or six primer pairs, as described above.

The term “comprising” as used in this specification means “consisting at least in part of”. When interpreting statements in this specification which include that term, the features, prefaced by that term in each statement or claim, all need to be present but other features can also be present. The related terms “comprises” and “comprised” are to be interpreted similarly.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE FIGURES

FIG. 1 shows the position of variable length regions and the flanking conserved regions that can be used for designing generic amplification primers. FIG. 1A shows the position of variable length regions in the Escherichia coli K12, strain MG1655 16S rRNA gene. FIG. 1A shows the position of variable length regions in the Escherichia coli K12, strain MG1655 23S rRNA gene.

FIG. 2 is an electropherogram showing multiplex PCR-generated amplicons from further pure cultures of Escherichia coli K12 strain MG1655.

FIG. 3 is an electropherogram showing multiplex PCR-generated amplicons from pure cultures of Lactobacillus gasseri DSM 20243 (type strain).

FIG. 4 is an electropherogram showing multiplex PCR-generated amplicons from pure cultures of Staphylococcus aureus ATCC 25923 (type strain).

FIG. 5 is an electropherogram showing the amplicon profile obtained from a high vaginal swab taken from a female subject with Bacterial Vaginosis.

FIG. 6 is an electropherogram showing the amplicon profile obtained from a high vaginal swab taken from a healthy female subject.

FIG. 7 is an electropherogram showing the profile obtained from a swab taken from a patient with an ear infection.

FIG. 8 is an electropherogram showing the amplicon profile obtained from a urine samples from a patient with a bladder infection.

FIG. 9 is an electropherogram showing the profile obtained from a tissue biopsy taken from the spine of a patient.

FIG. 10 is an electropherogram showing the profile generated from sputum obtained form a patient with cystic fibrosis.

### DETAILED DESCRIPTION

Methods for the identification of bioagent(s), such as bacteria, present in a sample using a combination of multiplex nucleic acid amplification, preferably multiplex PCR, followed by amplicon length determination are described herein. The primers described herein hybridize to conserved sequence regions of nucleic acids derived from bacteria and bracket variable sequence regions that uniquely identify the bacteria to a level of discrimination that is useful for the purpose of diagnosis or for determining the presence of bacteria in non-diagnostic settings, such as environmental monitoring and the like.

Amplicon detection is preferably mediated using one or more detectable labels, coupled with a size determination, such as gel electrophoresis, capillary electrophoresis, nanopore analysis or sequencing, or any other technique for separating amplicons on the basis of size. In certain embodiments, such techniques exclude mass spectrometry. Use of the primers described herein yields an amplicon profile which can be compared with a database of amplicon profiles for the same amplified regions. In preferred embodiments, the present method combines multiplex PCR-based amplification technology (which provides specificity) and amplicon size determination for bacteria detection and identification. In some embodiments, bioagent identity can be confirmed by use of other primer pairs, in addition to those described above. Other primer pairs may be chose for a particular application depending on the target bioagent. Examples of additional primer pairs include primer pairs that will amplify species specific sequences, toxin sequences, plasmid sequences, and phage sequences, for example.

The present method allows extremely rapid and accurate detection and identification of bacteria compared to existing methods. Furthermore, this rapid detection and identification is possible even when sample material is impure or contains multiple organisms. Thus, the method is useful in a wide variety of fields, including, but not limited to, forensics, basic research, food and beverage industries, regulatory monitoring programs, veterinary monitoring or diagnostics, equine monitoring or diagnostics, agricultural monitoring and testing, horticultural monitoring and testing, livestock monitoring and diagnosis, clinical identifications and medical diagnosis (including diagnosis of infectious diseases and conditions, such as diagnosis in conjunction with drug resistance detection), environmental testing (e.g., detection and discrimination of pathogenic vs. non-pathogenic bacteria in water or other samples), germ warfare (allowing immediate identification of the bacteria and appropriate treatment), biosecurity monitoring and any other application where the identification of bacteria to a useful level of discrimination is required. The method provides greatly improved sensitivity, specificity, reliability and rapidity compared to existing methods, with lower rates of false positives.

The present method can be used to detect and classify biological agents having 16S and 23S rRNA, or other sequences sufficiently similar to hybridise to the primers provided herein, such as bacteria. As one example, where the agent is an infectious disease-causing agent, the information obtained is used to determine practical information needed for treatment and prophylaxis.

The regions used for the oligonucleotide primer binding sites are conserved across all of the known divisions (kingdoms) of the Bacteria domain and so the method we describe is applicable for the detection of bacterial species found in a diverse range of environments and ecosystems. It is expected that the primers described herein or variants of primers falling within the conserved regions described herein will be applicable for known bacterial 16S and 23S rRNA genes described in public domain databases and bacteria that will be discovered in the future. Examples of such databases are The Ribosomal Database Project II (RDP; http://rdp.cme.msu.edu) and GenBank at the National Center for Biotechnology Information (NCBI: www.ncbi.nlm.nih.gov).

Accordingly, one aspect provides a method for the identification of a bioagent such as a bacterium present in a sample wherein the method includes at least the steps of:
contacting nucleic acid present in said sample with:
   a first pair of primers designed to produce a first amplicon from a 16S rRNA gene under amplification conditions;
   a second pair of primers designed to produce a second amplicon from a 16S rRNA gene or a 23S rRNA gene under amplification conditions;
   a third pair of primers designed to produce a third amplicon from a 23S rRNA gene under amplification conditions; and
   amplifying said nucleic acid with said first, second and third pair of primers;
determining the length of the amplicons produced; and
identifying the bioagent on the basis of the length of the amplicons.

Various methods are described above. The present method generates an amplicon profile for each bioagent. The advantage of amplicon profiling is that the amplicon profiles can be quantitatively generated in a massively parallel fashion using multiplex PCR (PCR in which two or more primer pairs amplify target sequences simultaneously) and size determination. These amplicon profiles uniquely identify bacteria of interest to a useful level of discrimination.

A “bioagent” is any organism, living or dead, or a nucleic acid derived from such an organism, having 16S and 23S rRNA. Samples may be alive or dead or in a vegetative state (for example, vegetative bacteria or spores) and may be encapsulated or bioengineered.

The term “16S rRNA” refers to the Ribosomal RNA small subunit (SSU rRNA) and includes small subunit rRNAs found in other bacteria that are lesser or greater Svedberg units in size, such as found in some species of Thermoanaerobacter or mitochondrial 16S-like rRNA. Accordingly, the term “16S rRNA gene” and grammatical equivalents refers to a DNA sequence or sequences encoding the 16S rRNA molecule.

Likewise, the term “23S rRNA” refers to the Ribosomal rRNA large subunit (LSU rRNA) and includes large subunit rRNAs found in other microorganims that are lesser or greater Svedberg units in size such as mitochondrial 23S-like rRNA.

As used herein, an amplicon profile is data representative of the length of each amplicon obtained or obtainable from amplification of bioagent nucleic acid using the primers described herein. As such, an amplicon profile can be thought of as a unique identifier of a bioagent to a useful level of discrimination.

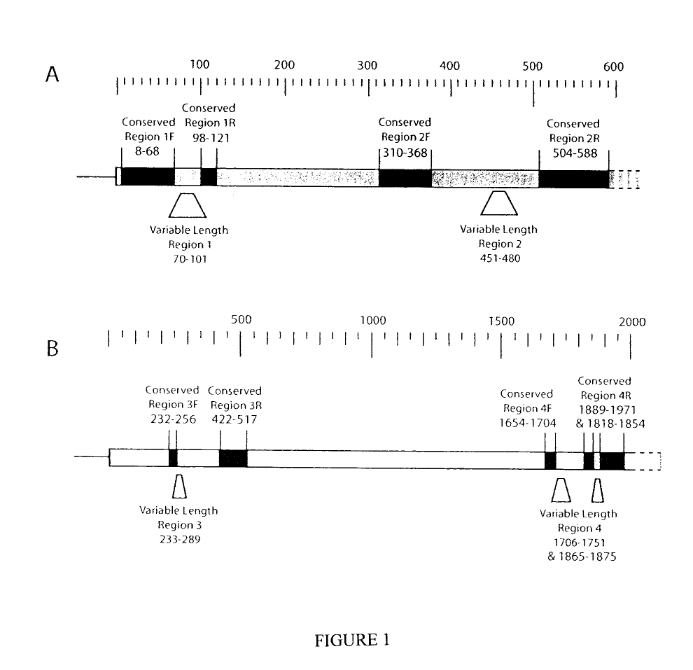

The primers used herein bind to partially conserved 16S or 23S rRNA gene sequence regions which flank an intervening region that varies in length between species. These sequence regions which flank the variable length region are highly conserved among many different species of bacterium. By the term highly conserved, it is meant that the sequence regions exhibit between about 80-100%, more preferably between about 90-100% and most preferably between about 95-100% identity. Examples of primers which amplify regions of the 16S or 23S rRNA are provided herein as SEQ ID NO. 1-28. Typical primer amplified regions in rRNA are shown in FIG. 1.

In one embodiment, four such sites are chosen: two in the 16S rRNA gene and two in the 23S rRNA gene. FIG. 1 shows the locations of these regions on the 16S rRNA gene (FIG. 1A) and 23S rRNA gene (FIG. 1B) of Escherichia coli K12 strain MG1655. The coordinates provided in FIG. 1 will be different for the corresponding genes in other species. The coordinates in these other species can be determined by performing a sequence alignment with the genes of Escherichia coli K12 strain MG1655.

By way of example, primers that amplify from these regions are provided in the following list and are also described in more detail below. It is understood that other primers can be used with different redundancies and in regions proximal to the coordinate provided, but bounded within the conserved regions shown in FIG. 1. The coordinates refer to the 16S rRNA and 23S rRNA genes of Escherichia coli K12 strain MG1655. Note also from FIG. 1B, two alternative conserved regions will work equally well—these regions correspond to the regions bordering Variable Region 4 of Escherichia coli K12 strain MG1655 23S rRNA gene at coordinates: 1706-1751 and 1865-1875.

### a) Examples of Amplicon I Primers.

| | |
|---|---|
| Forward | |
| MF1F | |
| 5′ AGR GTT TGA TCM TGG CTC AG 3′ | (SEQ ID NO: 1) |
| (Matches 16S positions 8-27) | |
| Reverse | |
| MF1R | |
| 5′ (FAM) GTT ACT CRC CCG TKC GCC 3′ | (SEQ ID NO: 3) |
| (Matches 16S positions 120-103) | |

b) Examples of Amplicon II primers.

| | |
|---|---|
| Forward | |
| MF2F | |
| 5′ (FAM) TCCTAC GGG AGG CAG CAG 3′ | (SEQ ID NO: 12) |
| (Matches 16S positions 339-356) or | |
| Reverse | |
| MF2R | |
| 5′ TGC TGG CAC GKA GTT AGC 3′ | (SEQ ID NO: 14) |
| (Matches 16S positions 522-504) | |

### c) Examples of Amplicon III Primers.

Forward

(SEQ ID NO: 17)

| | |
|---|---|
| MF3F | 5′ (FAM) GAT WYC SII AGT AGT GGC GAG CGA A 3′ |
| | (Matches 23S positions 232-256) |

Reverse

(SEQ ID NO: 19)

| | |
|---|---|
| MF3R | 5′ CTT TTC GCC KTT CCT TCA CGG TA 3′ |
| | (Matches 23S positions 481-459) |

### d) Examples of Amplicon IV Primers.

Forward

(SEQ ID NO: 21)

| | |
|---|---|
| MF4F | 5′ (HEX) TTS TIG TTA AGG AAC TMK GCA A 3′ |
| | (Matches 23S positions 1655-1676) |
| Reverse | |

(SEQ ID NO: 26)

| | |
|---|---|
| MF4R | 5′ CAA GGA ATT TCG CTA CCT TAG G 3′ |
| | (Matches 23S positions 1946-1925) |

One main advantage of the detection methods described herein is that the primers need not be specific for a particular bacterial species, or even genus, such as Bacillus or Streptomyces. Instead, the primers recognize highly conserved regions across most bacterial species including, but not limited to, the species described herein. Thus, the same set of primer pairs can be used to identify any desired bacterium because it will bind to the conserved regions which flank a variable length region specific to a single species, or common to several bacterial species, allowing nucleic acid amplification of the intervening sequence and determination of amplicon size. For example, the regions described above, including regions 8-27, 103-120, 339-356, and 504-522 (where numbers indicate nucleotide position) of bacterial 16S rRNA are highly conserved. Similarly, the regions described above, including regions 232-256, 459-481, 1655-1676, and 1925-1946 of bacterial 23S rRNA are also highly conserved. In one embodiment=, primers used in the present method bind to one or more of these regions or portions thereof.

Although the use of PCR is preferred, other nucleic acid amplification techniques may also be used, including linear amplification methods and isothermal amplification methods, for example but not limited to strand displacement amplification (SDA) or rolling circle amplification (RCA). Furthermore, the synthesis of a single-strand to generate a fragment spanning the variable length region can be detected by nanopore technology and so this method could be deemed as having undergone minor amplification—i.e. just a single copying. The degree of amplification required, will depend on the detection techniques used. For example, capillary electrophoresis may require more amplification that nanopore analysis. Similarly, although gel electrophoresis coupled with fluorescent detection is preferred, other methods of determining amplicon size may be utilised. The amplicon profile generated by multiplex amplification is then matched against amplicon profiles for one or more known bioagents. Preferably, the bioagent amplicon profile is matched against one or more databases of amplicon profiles from a large number of bioagents. While matching may be performed manually, preferably the matching is automated, for example the matching is done using a matching algorithm.

### 1 Primer Design

It is understood that the taxonomic groupings, for example class, family, order, genus, species and strain are ill-defined in bacteria and that these classifications are taxonomic and as such only partially correlate with phylogenic (evolutionary) based differentiation of organisms that uses DNA sequences. Hence the method, being phylogenetic in principle, will have different levels of taxonomic discrimination with different organisms. This is not a reflection of inconsistency in the method but of inconsistency in microbial taxonomic classifications. For example, different taxonomic strains of Escherichia coli have been shown to differ by as much as 30% at the genomic level while Bacillus anthracis, thuringiensis and cereus, differ by little more than transferable plasmids.

It is also understood that many bacterial species will produce a similar pattern when using this method. This limitation is greatly alleviated for a number of reasons: 1. Only a limited subset of bacteria can be expected to be present in any sample. For example, the many million environmental species of bacteria will not be found in clinical samples with the exception of only a few opportunistic pathogens. 2. For many purposes, it is not necessary to classify an organism with great precision. The usefulness of this invention is in its ability to provide a generic method that discriminates to a level that captures functionality (for example pathogenicity) of an organism and thereby provides information that can be acted upon.

It is understood that primer variants can be designed within the conserved regions used in combination as primer binding sites (FIG. 1). Variations in primers can be in the form of alternative bases at one or more sites, alternative methods for dealing with non-conserved sites, alternative primer lengths and finally, alternative positioning of the primer binding sites within the conserved regions. It is well known that A pairs with T, while G pairs with C, but non-canonical pairing can also occur between G and T while still providing a favourable Gibbs free energy thereby increasing primer binding stability. On this basis, it is possible to use a ‘K’ (G or T) in a primer to act as a redundancy capable of binding to all nucleotides on the complementary strand. This approach only increases the plurality of oligonucleotides in the mixture by two rather than three or four if multiple redundancies are designed into the primer. Alternatively an inosine or an abasic linker can be used at the variable nucleotides.

5′ Fluorescent labels or can be added to the forward or the reverse primer in each pair although it is understood that a preferential choice of primer for labelling will be: Amplicon I, reverse; Amplicon II, forward; Amplicon III, reverse; Amplicon IV, forward. The reason for this preference is to render contiguous amplification of amplicons to be unlabelled. It is also understood that modified blocking oligonucleotides for example Peptide Nucleic Acids (PNAs), or Locked Nucleic Acids (LNAs) or other such high affinity nucleic acid binding agents can be used to reduce this eventuality.

The following tables 1 to 9 provide the preferred primer set used in the examples presented in this patent application and also examples of alternative oligonucleotide primers that will function as primers in the PCR or other DNA amplification method. In addition, the tables describe the most favourable region of DNA from which the primers can be designed. It is understood that these alternative primers do not represent a complete list of possible variants but are preferred embodiments. The database accession number of the complete genome of E. coli K12 is NC_{—}000913 and the nucleotide sequence is incorporated herein by reference in its entirety. The nucleotide sequence of E. coli K12 16S rRNA is provided herein as SEQ ID No. 30 and the nucleotide sequence of E. coli K12 23S rRNA is provided herein as SEQ ID No. 29.

The nucleotide symbols used are standard nomenclature (M=A or C, R=A or G, W=A or T, S═C or G, Y═C or T, K=G or T, N=A, G, C or T, and I=Inosine). All coordinates are based on the gene sequences from E. coli K12, strain MG 1566.

### Amplicon I

TABLE 1

Forward: Conserved region coordinates: 8-68
(FIG. 1A)

| | | |
|---|---|---|
| | | SEQ |
| | | ID |
| Primer | Sequence | No. |

| | | | | |
|---|---|---|---|---|
| MF1F | AGRGTTTGATCMTGGCTCAG | 8 | 27 | 1 |
| MF1F- | AGRRTTYGATCMTGGYTCAGRNYKAACGCTGGCGGC | 8 | 43 | 2 |
| con | * | | | |

*Any shorter primer encompassed within these 36 nucleotides of sequence.

TABLE 2

Reverse: Conserved region coordinates: 98-121
(FIG. 1A)

| | | | | |
|---|---|---|---|---|
| MF1R | GTTACTCRCCCGTKCGCC | 121 | 104 | 3 |
| MF1aR | GTTACTCRCCCGTICGCC | 121 | 104 | 4 |
| MF1bR | GTTACTCRCCCGTKCGC | 121 | 105 | 5 |
| MF1cR | GTTACTCRCCCGTICGC | 121 | 105 | 6 |
| MF1dR | TTACTCRCCCGTKCGCC | 120 | 104 | 7 |
| MF1eR | TTACTCRCCCGTICGCC | 120 | 104 | 8 |
| MF1fR | TTACTCRCCCGTKCGC | 120 | 105 | 9 |
| MF1gR | TTACTCRCCCGTICGC | 120 | 105 | 10 |
| MF1R-con | GTTACTYRCCCNTNCRCY* | 121 | 104 | 11 |

*Any shorter primer encompassed within these 18 nucleotides of sequence.

### Amplicon II

TABLE 3

Forward: Conserved region coordinates: 310-368
(FIG. 1A)

| | | | | |
|---|---|---|---|---|
| MF2F | TCCTACGGGAGGCAGCAG | 340 | 357 | 12 |
| MF2-con | ACTCCTACGGGAGGCAGCAGT NGRGAAT* | 338 | 365 | 13 |

*Any shorter primer encompassed within these 28 nucleotides of sequence.

TABLE 4

Reverse: Conserved region coordinates: 504-588
(FIG. 1A)

| | | | | |
|---|---|---|---|---|
| MF2R | TGCTGGCACGKAGTTAGC | 523 | 506 | 14 |
| MF2R- | YGTATYACCGCRRCTGCTGGCACRNAGTTRGYCG | 537 | 504 | 15 |
| con | * | | | |

*Any shorter primer encompassed within these 34 nucleotides of sequence.

### Amplicon III

TABLE 5

Forward: Conserved region coordinates: 232-256
(FIG. 1B)

| | | | | |
|---|---|---|---|---|
| MF3aF | AGTAGTGGCGAGCGAA | 241 | 256 | 16 |
| MF23 | GATWYCSIIAGTAGTGGCGAGCGAA | 232 | 256 | 17 |
| MF3F-con | GATTNNNNNAGTAGTGGCGAGCGAA* | 232 | 256 | 18 |

*Any shorter primer encompassed within these 25 nucleotides of sequence.

TABLE 6

Reverse: Conserved region coordinates: 422-517
(FIG. 1B)

| | | | | |
|---|---|---|---|---|
| MF3R | CTTTTCGCCKTTCCTTCACGGTA | 481 | 459 | 19 |
| MF3R- | RGTNCTTTTCRCCNTTCCRTCACRGTACTNNTNC | 485 | 442 | 20 |
| con | NCTATCGNTN* | | | |

*Any shorter primer encompassed within these 44 nucleotides of sequence.

### Amplicon IV

TABLE 7

Forward: Conserved region coordinates: 1654-1704
(FIG. 1B)

| | | | | |
|---|---|---|---|---|
| MF4F | TTSTIGTTAAGGAACTMKGCAA | 1656 | 1677 | 21 |
| MF4aF | GTTAAGGAACTMKGCAA | 1661 | 1677 | 22 |
| MF4F-con | AAYNNNGNNAAGGAACTMKGCAA* | 1654 | 1677 | 23 |

*Any shorter primer encompassed within these 23 nucleotides of sequence.

TABLE 8

Reverse region 1: Conserved region coordinates:
1818-1854 (FIG. 1B)

| | | | | |
|---|---|---|---|---|
| MF2R | GCAYYGGGCAGGIGTCA | 1843 | 1827 | 24 |
| MF2R-con | TTAACCTTNNRGCAYYGGGCAGGYGTCA* | 1854 | 1827 | 25 |

*Any shorter primer encompassed within these 28 nucleotides of sequence.

TABLE 9

Reverse region 2: Conserved region coordinates:
1889-1971 (FIG. 1B)

| | | | | |
|---|---|---|---|---|
| MF2R | CAA GGA ATT TCC CTA CCT TAG G | 1945 | 1925 | 26 |
| MF2aR | CAA GGA ATT TCG CTA CCT TAG | 1945 | 1926 | 27 |
| MF2R- | CCWTTCRWGCRGGWCGGWAYTTACCCGACAA | 1971 | 1891 | 28 |
| con | GGAATTTCGCTACCTTAGGAYSGTTATAGTT | | | |
| | ACSRCCGCCRTTYACYNGGGCTT* | | | |

*Any shorter primer encompassed within these 85 nucleotides of sequence.

### 2 Polymerase Chain Reaction

The general principles and conditions for amplification and detection of nucleic acids using polymerase chain reaction (PCR) are well known, the details of which are provided in numerous references including U.S. Pat. Nos. 4,683,195 (Mullis et al.), 4,683,202 (Mullis), and 4,965,188 (Mullis et al.), all of which are incorporated herein by reference. Many variations of PCR exist, and may be suitable for use in the present method. Thus, in view of the teaching in the art and the specific teaching provided herein, a worker skilled in the art should have no difficulty in practicing the present method by co-amplifying three or more gene regions of bioagent(s) in accordance with the methods described herein.

The term “oligonucleotide” refers to a molecule composed of one or more deoxyribonucleotides or ribonucleotides, such as primers, probes, and nucleic acid fragments to be detected.

The term “primer” refers to an oligonucleotide, whether naturally occurring or synthetically produced, that is capable of acting as a point of initiation of synthesis when placed under conditions in which synthesis of a primer extension product complementary to a nucleic acid strand (that is, template) is induced, such conditions include the presence of other reagents, such as PCR reagents, and suitable temperature and pH.

The primer is preferably single stranded for maximum efficiency in amplification, but can contain a double stranded region if desired. It must be long enough to prime the synthesis of extension products in the presence of the DNA polymerase. The exact size of each primer will vary depending upon the use contemplated, the concentration and sequence of the primer, the complexity of the targeted sequence, the reaction temperature, and the source of the primer. Generally, the primers used will have from 12 to 60 nucleotides, and preferably, they have from 16 to 40 nucleotides. More preferably, each primer has from 18 to 35 nucleotides.

Primers useful herein can be prepared using known techniques and equipment, including for example an ABI DNA Synthesizer (available from Applied Biosystems) or a Biosearch 8600 Series or 8800 Series Synthesizer (available from Milligen-Biosearch, Inc.). Procedures for using this equipment are well known and described for example in U.S. Pat. No. 4,965,188 (Gelfand et al.), incorporated herein by reference. Naturally occurring primers isolated from biological sources may also be useful (such as restriction endonuclease digests).

As used herein, a “probe” is an oligonucleotide or polynucleotide which is substantially complementary to a nucleic acid sequence of the target nucleic acid and which is used for detection or capture of the amplified target nucleic acid.

Sequence specific primers and probes are described herein. It will be apparent to those skilled in the art that additional sequence specific primers and probes can be prepared by, for example, the addition of nucleotides to either the 5′ or 3′ ends, which nucleotides are complementary or noncomplementary to the target sequence.

The primers and/or the probes described herein can, optionally, be labeled. Using known methods in the art, the primers and/or probes can be labeled with a specific binding ligand (such as biotin or antibodies, such as antibodies available commercially from Millipore™, Invitrogen™, or eBioscience™), an enzyme (such as glucose oxidase, peroxidases, uricase, and alkaline phosphatase), radioisotopes, electron-dense reagents, chromogens, fluorogens or fluorophores, phosphorescent moieties, ferritin, nanodots or Q-dots, or any other type of label that can be used to differentiate amplicons. Preferably, the label is a fluorescent label, particularly those suitable for use in automated sequencers, such as FAM, HEX, JOE, ROX, TMARA, or TET.

A “PCR reagent” refers to any of the reagents considered essential for PCR, usually a set of primers for each target nucleic acid, a DNA polymerase (preferably a thermostable DNA polymerase), a DNA polymerase cofactor, and one or more deoxyribonucleoside-5′-triphosphates (dNTP's) or similar nucleosides. Other optional reagents and materials used in PCR are described below. These reagents can be provided individually, as part of a test kit, or in reagent chambers of test devices.

A DNA polymerase is an enzyme that will add deoxynucleoside monophosphate molecules to (usually the 3′-hydroxy) end of the primer in a complex of primer and template, but this addition is in a template dependent manner. Generally, synthesis of extension products proceeds in the 5′ to 3′ direction of the newly synthesized strand until synthesis is terminated. Useful DNA polymerases include, for example, Taq polymerase, E. coli DNA polymerase I, T4 DNA polymerase, Klenow polymerase, reverse transcriptase and others known in the art. Preferably, the DNA polymerase is thermostable meaning that it is stable to heat and preferentially active at higher temperatures, especially the high temperatures used for priming and extension of DNA strands. More particularly, thermostable DNA polymerases are not substantially inactive at the high temperatures used in polymerase chain reactions as described herein. Such temperatures will vary depending on a number of reaction conditions, including pH, nucleotide composition, length of primers, salt concentration and other conditions known in the art.

A number of thermostable DNA polymerases have been reported in the art, including those mentioned in detail in U.S. Pat. Nos. 4,965,188 (Gelfand et al.) and 4,889,818 (Gelfand et al.), both incorporated herein by reference. Particularly useful polymerases are those obtained from various Thermus bacterial species, such as Thermus aquaticus, Thermus thermophilus, Thermus filiformis, and Thermus flavus. Other useful thermostable polymerases are obtained from various microbial sources including Thermococcus literalis, Pyrococcus furiosus, Thermotoga sp. and those described in WO-A-89/06691 (published Jul. 27, 1989). Some useful thermostable polymerases are commercially available, such as, AmpliTaq™, Tth, and UlTma™ from Perkin Elmer, Pfu from Stratagene, and Vent and Deep-Vent from New England Biolabs. A number of techniques are also known for isolating naturally-occurring polymerases from organisms, and for producing genetically engineered enzymes using recombinant techniques.

A DNA polymerase cofactor refers to a nonprotein compound on which the enzyme depends for activity. Thus, the enzyme is catalytically inactive without the presence of cofactor. A number of materials are known cofactors including, but not limited to, manganese and magnesium salts, such as chlorides, sulfates, acetates and fatty acids salts. Magnesium chlorides and sulfates are preferred.

Also needed for PCR are two or more deoxyribonucleoside-5′-triphosphates, such as two or more of dATP, dCTP, dGTP, dTTP and dUTP. Analogues such as dITP and 7-deaza-dGTP are also useful. Preferably, the four common triphosphates (dATP, dCTP, dGTP and dTTP) are used together.

The PCR reagents described herein are provided and used in PCR in suitable concentrations to provide amplification of the target nucleic acid. The minimal amounts of primers, DNA polymerase, cofactors and deoxyribonucleoside-5′-triphosphates needed for amplification and suitable ranges of each are well known in the art. The minimal amount of DNA polymerase is generally at least about 0.5 units/100 μl of solution, with from about 2 to about 25 units/100 μl of solution being preferred, and from about 7 to about 20 units/100 μl of solution being more preferred. Other amounts may be useful for given amplification systems. A “unit” is defined herein as the amount of enzyme activity required to incorporate 10 nmoles of total nucleotides (dNTP's) into an extending nucleic acid chain in 30 minutes at 74° C. The minimal amount of primer is at least about 0.075 μMol with from about 0.1 to about 2 μMol being preferred, but other amounts are well known in the art. The cofactor is generally present in an amount of from about 2 to about 15 mMol. The amount of each dNTP is generally from about 0.25 to about 3.5 mMol.

The PCR reagents can be supplied individually, or in various combinations, or all in a buffered solution having a pH in the range of from about 7 to about 9, using any suitable buffer, many of which are known in the art.

Other reagents that can be used in PCR include, for example, antibodies specific for the thermostable DNA polymerase. Antibodies can be used to inhibit the polymerase prior to amplification. Preferably, the antibodies are specific for the thermostable DNA polymerase, inhibit the enzymatic activity of the DNA polymerase at temperatures below about 50° C., and are deactivated at higher temperatures. Useful antibodies include, monoclonal antibodies, polyclonal antibodies and antibody fragments. Preferably, the antibody is monoclonal. Antibodies can be prepared using known methods such as those described in Harlow et al., Antibodies: A Laboratory Manual, Cold Spring Harbor, N.Y. (1988).

Representative monoclonal antibodies are described in U.S. Pat. No. 5,338,671 (Scalice et al.), the contents of which are hereby incorporated by reference. Two such monoclonal antibodies are readily obtained by a skilled artisan using conventional procedures, and starting materials including either of hybridoma cell lines HB 11126 or 11127, deposited with the American Type Culture Collection (ATCC) (Rockville, Md.). The monoclonal antibody is present in an amount of from about 5:1 to about 500:1 molar ratio to the DNA polymerase.

A target nucleic acid, including that from a bioagent, can be obtained from any of a variety of sources such as peripheral blood mononuclear cells (PBMC's), whole blood, respiratory fluids, lymph, and stool. Generally, it is extracted in some conventional manner to make it available for contact with the primers and other PCR reagents. If the target nucleic acid is double stranded, the two strands must be separated before priming can occur. Denaturation can be accomplished using any of the known techniques such as heat treatment, physical treatment or chemical treatment.

Amplification is preferably conducted in a continuous, automated manner so that the reaction mixture is temperature cycled in a controlled manner for desired preset times. A number of instruments have been developed for this purpose and are available to those skilled in the art. Preferably, amplification is carried out in a closed reaction vessel, such as the chemical test pack described in U.S. Pat. No. 5,229,297, which vessel is processed on the instrument described in U.S. Pat. No. 5,089,233.

Amplified nucleic acids can be detected in a number of known ways, such as those described in U.S. Pat. No. 4,965,188 (Gelfand et al.). For example, the amplified nucleic acids can be detected using Southern blotting, dot blot techniques, or nonisotopic oligonucleotide capture detection with a labelled probe. Alternatively, amplification can be carried out using primers that are appropriately labelled, and the amplified primer extension products can be detected using procedures and equipment for detection of the label.

In a preferred embodiment, the amplified target nucleic acid is detected using an oligonucleotide probe that is labelled for detection and can be directly or indirectly hybridized with the amplified target. In another preferred embodiment, one or more of the primers used to amplify the target nucleic acid is labeled, for example, with a specific binding moiety. The resulting primer extension product into which the labeled primer has been incorporated can be captured with a probe. Detection of the amplified target hybridized to the probe can be achieved by detecting the presence of the labeled probe or labeled amplified target using suitable detection equipment and procedures that are well known in the art. Certain labels may be visible to the eye without the use of detection equipment.

In a more preferred embodiment, one or more of the primers used to amplify the target nucleic acid is labeled with a fluorescent label. More preferably, at least one primer of each primer pair is labeled with a different fluorescent label, so that each amplicon produced on amplification is labeled with a different label. These may then be detected, for example using an automated sequencer. See the examples presented herein for exemplary methods using such labeled primers.

Preferably, amplification and detection are carried out in a closed reaction vessel to reduce the risk of contamination. Both amplification and detection can be carried out in a closed reaction vessel as described in U.S. Pat. No. 5,229,297, without opening up the reaction vessel during the process.

As used herein, when in reference to time the term “about” refers to +/−10% of that time limit. When used in reference to temperatures, the term “about” refers to +/−5° C.

### 3 Polynucleotide Variants

The term “variant” as used herein refers to polynucleotide sequences different from the specifically identified sequences, wherein one or more nucleotides is deleted, substituted, or added. Variants may be naturally occurring allelic variants, or non-naturally occurring variants. Variants may be from the same or from other species and may encompass homologues, paralogues and orthologues. In certain embodiments, variants of the polynucleotides possess biological activities that are the same or similar to those of the wild type polynucleotides. The term “variant” with reference to polynucleotides encompasses all forms of polynucleotides as defined herein.

Variant polynucleotide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified polynucleotide sequence. Identity is found over a comparison window of at least 20 nucleotide positions, preferably at least 50 nucleotide positions, at least 100 nucleotide positions, or over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.10 [October 2004]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters: bl2seq-i nucleotideseq1-j nucleotideseq2-F F-p blastn

The parameter −F F turns off filtering of low complexity sections. The parameter −p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.10 [October 2004]) from NCBI (ftp://ftp.ncbi.nih.gov/blast/).

The similarity of polynucleotide sequences may be examined using the following unix command line parameters: bl2seq-i nucleotideseq1-j nucleotideseq2-F F-p tblastx

The parameter −F F turns off filtering of low complexity sections. The parameter −p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

Alternatively, variant polynucleotides hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule in an amplification reaction mixture) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tₘ=81. 5+0.41% (G+C-log(Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides also encompass polynucleotides that differ from the sequences described herein as a consequence of the degeneracy of the genetic code. A sequence alteration that does not change the proper formation of ribosomal RNA is a “silent variation”. Nucleotides may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.10 [October 2004]) from NCBI (ftp://ftp.ncbi.nih.goviblast/) via the tblastx algorithm as previously described.

### 4 Determination of Amplicon Size

Following the generation of amplicons, the methods described herein require that the size of the amplicons be determined so as to identify the bioagent. A number of method for determining the size of nucleic acid molecules exist and are well known in the art.

Electrophoresis, particularly capillary electrophoresis such as that performed during automated sequencing (for example, on an ABI 3100 Genetic Analyzer or equivalent), is particularly suitable for the methods described herein. Internal size standards can be employed, allowing for highly accurate size determination. When coupled with fluorescent labelling of primers, particularly differential labelling of primer pairs, rapid identification of bioagents is possible. Examples of such analyses are presented herein.

While the above automated methods for amplicon size determination are preferred, it will be appreciated that any method capable of determining the size of amplicons with sufficient accuracy will be suitable for use in the methods described herein. Such methods may include mass spectroscopy, spectrometry or nanopore analysis etc, although in certain embodiments mass spectroscopy is excluded. Preferably, such methods can discriminate single nucleotide differences in amplicon length.

### 5 Diagnostic Kits

Diagnostic kits useful for identifying bioagents, for example, for practicing the methods described herein are also provided.

Accordingly, one embodiment provides a diagnostic kit which can be used to determine the identity of a bioagent. In one embodiment, the diagnostic kit contains a oligonucleotide primer having or containing the sequence shown in any one of SEQ ID No: 1 to 28. One kit includes a set of primers or primer pairs as described herein. The primers may be used in conventional hybridisation, Taqman assays, OLE assays, etc. Alternatively, primers can be designed to permit identification by microsequencing.

One kit of primers can include first, second and third primer pairs as described herein. Optionally, such a kit may include a fourth, fifth or sixth primer pairs. Preferably the kit includes instructions for use, for example in accordance with the methods described herein.

In other embodiments, the kit may include one or more nucleotide probes, for example, for hybridisation with nucleic acid from a sample containing bioagent or with an amplicon generated by a method described herein, and means for detecting the nucleotide probe bound to nucleic acid in the sample, optionally together with a standard. In a particular aspect, the kit for practicing the methods described herein includes a probe having a nucleic acid molecule sufficiently complementary with a sequence present in an amplicon generated by the methods described herein, so as to bind thereto under stringent conditions. “Stringent hybridisation conditions” takes on its common meaning to a person skilled in the art. Appropriate stringency conditions which promote nucleic acid hybridisation, for example, 6× sodium chloride/sodium citrate (SSC) at about 45° C. are known to those skilled in the art, including in Current Protocols in Molecular Biology, John Wiley & Sons, NY (1989). Appropriate wash stringency depends on degree of homology and length of probe. If homology is 100%, a high temperature (65° C. to 75° C.) may be used. However, if the probe is very short (<100 bp), lower temperatures must be used even with 100% homology. In general, one starts washing at low temperatures (37° C. to 40° C.), and raises the temperature by 3-5° C. intervals until background is low enough to be a major factor in autoradiography. The diagnostic kit can also contain an instruction manual for use of the kit.

The kit can also include a buffering agent, a preservative, or a protein stabilizing agent. The kit can also include components necessary for detecting the detectable agent (e.g., an enzyme or a substrate). The kit can also contain a control sample or a series of control samples which can be assayed and compared to the test sample contained. The kit may also include reagents for the isolation or preparation of nucleic acid from the sample, including proteinases, chaotropic agents, osmotically active agents, wash buffers, and the like, particularly those components described herein in the examples. Each component of the kit can be enclosed within an individual container and all of the various containers can be within a single package, along with instructions for interpreting the results of the assays performed using the kit.

### 6 Sample Preparation

As will be apparent to persons skilled in the art, samples suitable for use in the methods described herein may be obtained from the environment (such as soil, rock, water and plant material samples, for example) or from subjects, including tissues or fluids from a subject, as convenient, and so that the sample contains the bioagent(s) to be tested.

Biological samples can be obtained from a wide range of substrates including clinical, food and beverage or environmental samples. Typically, microbial samples are obtained from environmental sources and for food testing by either taking a sample of a liquid or solid under tests or by swabbing a solid surface.

Conveniently, clinical samples may be taken from tissues, blood, serum, plasma, cerebrospinal fluid, urine, semen, swabs or saliva. Tissue samples may be obtained using standard techniques such as cell scrapings or biopsy techniques to collect tissue animal subjects. Similarly, blood sampling is routinely performed, for example for pathogen testing, and methods for taking blood samples are well known in the art. Likewise, methods for storing and processing biological samples are well known in the art. For example, tissue samples may be frozen until tested if required. In addition, one of skill in the art would realize that some test samples would be more readily analyzed following a fractionation or purification procedure, for example, separation of whole blood into serum or plasma components.

### 6.1 Bacteria Present in Samples Containing Large Quantities of Pus (Immune Cells)

The presence of animal immune response cells (macrophages and neutrophils), which have been recruited to sites of bacterial invasion can be problematic for bacterial identification. Some of the bacteria may be phagocytosed and inside the immune cells; the immune cells represent the bulk of material and protein present in the sample; animal cells contain mitochondrial 16S type rRNA genes; and there is probably some haem from red blood cells and myoglobin (particularly from biopsy material) which inhibits subsequent PCR.

Routine methods to isolate DNA from such samples may not yield robust amplification. For example, the use of bead beating protocols to smash cells may result in poor but discernable PCR products. It is therefore useful to at least partially purify bacterial samples containing pus. Most of the pus cells are removed by slow centrifugation, leaving bacteria in the supernatant, as described herein in the Examples. Proteinase treatment can also be used. Also, the small amount of crude bacterial DNA usually obtained may be precipitated and resuspended in small volume to concentrate it prior to PCR.

### 6.2 Bacterial Capsules

Some bacteria, particularly when growing on rich medium, secrete a thick capsule—the composition of which varies from one species to another. It is likely that similar encapsulation occurs in biofilms. When such encapsulated bacteria are treated with normal preparatory methods, typically no DNA is released.

In such cases, bacterial lysis and/or DNA isolation can be improved using a an agent that damages the capsule sufficiently so that the proteinase became effective on the cells after washing.

### 6.3 Osmotically Fragile Bacteria Lacking Cell Walls

Some samples may contain a mixed microflora which includes bacteria lacking cell walls. Examples of such organisms of clinical significance are Ureaplasma spp., Mycoplasma spp., etc. These cells are fragile, and in hypotonic solutions lyse rapidly and their DNA can be lost during initial cell washing steps. Similarly, if a bead beating technology is used to break cells, bacteria lacking walls are broken so quickly that their DNA is damaged in the ensuing glass bead grinding and is not amplified in subsequent DNA amplification steps. Thus these pathogenic bacteria often remain undiagnosed and unidentified using molecular analyses involving initial cell breakage. They are usually not seen by microscopy, and their isolation on agar medium is highly skilled and often unsuccessful. Known techniques for handling such organisms can be used.

### 6.4 Unknown, Unculturable, or Senescent Bacteria and Mixed Cultures

When a sample of infected material, for example a vaginal swab from a subject with BV, is investigated, it is not uncommon to find unknown bacteria, unculturable bacteria, senescent bacteria, mixed cultures of bacteria, or mitochondrial 16S type DNA.

Unknown and unculturable bacteria can be placed in taxonomic groups by amplifying their 16S rRNA gene, and subsequently their signature amplicon pattern can be recognized if they are encountered again. Senescent bacteria can likewise be identified from amplified 16S rRNA gene sequence. Mixed cultures are reasonably easy to identify, because there are several peaks in each amplicon, and these can readily be grouped and/or differentiated. Examples showing the identification of multiple bacterial species present in mixed culture are presented herein. The mitochondrial 16S rRNA gene, which can be problematic in some methodologies, is not so using the methods described herein because its sequence is known, and the amplicon pattern (for 16S sizes) can be recognized and discounted. Alternatively, specific high affinity oligonucleotide variants can be added to specifically inhibit the amplification of such unwanted amplicons.

### 7 Computer-Related Embodiments

It will also be appreciated that the methods described herein are amenable to use with and the results analysed by computer systems, software and processes. Computer systems, software and processes to identify and analyse nucleic acid data are well known in the art. For example, the results of one or more genetic analyses as described herein may be analysed using a computer system and processed by such a system.

The results of an analysis of the amplicons generated by and utilised in the methods described herein may be “provided” in a variety of mediums to facilitate use thereof. As used in this section, “provided” refers to a manufacture, other than an isolated nucleic acid molecule, that contains amplicon information. Such a manufacture provides the amplicon information in a form that allows a skilled artisan to examine the manufacture using means not directly applicable to examining the amplicons or a subset thereof as they exist in nature or in purified form. The amplicon information that may be provided in such a form includes any of the amplicon information provided by the methods described herein such as, for example, amplicon length, bioagent identity, bioagent information, and the like.

In one application of this embodiment, the amplicon information and the results of an analysis of the amplicons utilised in the present methods can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture having computer readable medium having recorded thereon amplicon information. One such medium is provided with the present application, namely, the present application contains computer readable medium (floppy disc) that has nucleic acid sequences used in analysing the amplicons utilised in the methods described herein provided/recorded thereon in ASCII text format in a Sequence ID Listing.

As used herein, “recorded” refers to a process for storing information on computer readable medium. A skilled artisan can readily adopt any of the presently known methods for recording information on computer readable medium to generate manufactures having the amplicon information.

A variety of data storage structures are available to a skilled artisan for creating a computer readable medium having recorded thereon amplicon information. The choice of the data storage structure will generally be based on the means chosen to access the stored information. In addition, a variety of data processor programs and formats can be used to store the amplicon information on computer readable medium. For example, sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (for example, text file or database) in order to obtain computer readable medium having recorded thereon the amplicon information.

By providing the amplicons and/or the results of an analysis of the amplicons utilised in the methods described herein in computer readable form, a skilled artisan can routinely access the amplicon information for a variety of purposes. Computer software is publicly available which allows a skilled artisan to access sequence information provided in a computer readable medium. Examples of publicly available computer software include BLAST (Altschul et al, J. Mol. Biol. 215:403-410 (1990)) and BLAZE (Brutlag et al, Comp. Chem. 17:203-207 (1993)) search algorithms.

Another embodiment further provides systems, particularly computer-based systems, which contain the amplicon information described herein. Such systems may be designed to store and/or analyze information on, for example, a number of amplicon lengths, or information on amplicon lengths from a number of bioagents. The amplicon information represents a valuable information source. The amplicon information stored/analyzed in a computer-based system may be used for such applications as identifying or selecting bioagents, in addition to computer-intensive applications as determining or analyzing amplicon lengths for various bioagents, correlating amplicon lengths with unknown bioagents, or for various other bioinformatic, pharmacogenomic, drug development, or selection or identification applications.

As used herein, “a computer-based system” refers to the hardware, software, and data storage used to analyze the amplicon information. The minimum hardware of the computer-based systems typically includes a central processing unit (CPU), an input, an output, and data storage. A skilled artisan can readily appreciate that any one of the currently available computer-based systems are suitable for use in the methods described herein. Such a system can be changed into a system of use in the methods described herein by utilizing the amplicon information, such as that provided herewith on the floppy disc, or a subset thereof, without any experimentation.

As stated above, the computer-based systems have data storage having stored therein amplicon information, such as amplicon lengths and/or the results of an analysis of the amplicons utilised in the methods described herein, and the necessary hardware and software for supporting and implementing one or more programs or algorithms. As used herein, “data storage” refers to memory which can store amplicon information, or a memory access facility which can access manufactures having recorded thereon the amplicon information.

The one or more programs or algorithms are implemented on the computer-based system to identify or analyze the amplicon information stored within the data storage. For example, such programs or algorithms can be used to determine the expected amplicon length in a target sequence or bioagent, or to analyse the results of a genetic analysis of the amplicons described herein. As used herein, a “target sequence” can be any DNA sequence containing the amplicon to be analysed, searched or queried, while “target bioagent” can be any bioagent to be identified.

A variety of structural formats for the input and output can be used to input and output the information in the computer-based systems. An exemplary format for an output is a display that depicts the amplicon information, such as the amplicon length. Such presentation can provide a rapid, binary scoring system for many amplicons or bioagents simultaneously. It will be appreciated that such output may be accessed remotely, for example over a LAN or the internet. Typically, given the nature of amplicon information, such remote accessing of such output or of the computer system itself is available only to verified users so that the security of the amplicon information and/or the computer system is maintained. Methods to control access to computer systems and the data residing thereon are well-known in the art, and are amenable to the embodiments.

One exemplary embodiment of a computer-based system having amplicon information that can be used to implement the present method includes a processor connected to a bus. Also connected to the bus are a main memory (preferably implemented as random access memory, RAM) and a variety of secondary storage devices, such as a hard drive and a removable medium storage device. The removable medium storage device may represent, for example, a floppy disc drive, a CD-ROM drive, a magnetic tape drive, etc. A removable storage medium (such as a floppy disc, a compact disc, a magnetic tape, etc.) containing control logic and/or data recorded therein may be inserted into the removable medium storage device. The computer system includes appropriate software for reading the control logic and/or the data from the removable storage medium once inserted in the removable medium storage device. The amplicon information may be stored in a well-known manner in the main memory, any of the secondary storage devices, and/or a removable storage medium. Software for accessing and processing the amplicon information (such as amplicon scoring tools, search tools, comparing tools, etc.) preferably resides in main memory during execution.

Accordingly, a system for performing one or more of the methods described herein is provided. The system includes:

computer processor means for receiving, processing and communicating data;

storage means for storing data including a reference genetic database of the results of analysis of one or more bioagents; and

a computer program embedded within the computer processor which, once data consisting of or including the result of a analysis for which data is included in the reference genetic database is received, processes said data in the context of said reference databases to determine, as an outcome, the identity of the bioagent, said outcome being communicable once known, preferably to a user having input said data.

Preferably, said system is accessible via the internet or by personal computer.

Preferably, the genetic analysis includes the steps of contacting nucleic acid present in said sample with:
a first pair of primers designed to produce a first amplicon from a 16S rRNA gene under amplification conditions;
a second pair of primers designed to produce a second amplicon from a 16S rRNA gene or a 23S rRNA gene under amplification conditions;
a third pair of primers designed to produce a third amplicon from a 23S rRNA gene under amplification conditions; and
amplifying said nucleic acid with said first, second and third pair of primers; and determining the length of the amplicons produced.

Suitable methods are described in more detail above. More preferably, the reference genetic database includes the length of the amplicons associated with at least one bioagent.

In yet a further aspect, an embodiment provides a computer program suitable for use in a system as defined above, including a computer usable medium having program code embodied in the medium for causing the computer program to process received data consisting of or including the result of at least one genetic analysis of one or more bioagents, in the context of a reference genetic database of the results of at least one genetic analysis.

The invention consists in the foregoing and also envisages constructions of which the following gives examples only.

### EXAMPLES

### Example 1

### DNA Extraction from Bacterial Colonies (Escherichia coli)

### Introduction

This experiment describes the extraction of DNA from Escherichia coli K12, strain MG1655 colonies on agar plates and its use in the identification of the bacteria using the methods described herein.

### Reagents

20 mM HEPES buffer pH 7.7
Lysozyme at 5 mg/ml
prepGEM™ protease at 1 U/ml (ZyGEM, New Zealand)
prepGEM™ 5×ZyGEM Buffer 4 (ZyGEM, New Zealand)

### Isopropanol

Glycogen (20 μg/μL)

### Methods

### 1. DNA Isolation

1. Pick up 1-2 bacterial colonies from plate and wash in buffer.
2. Concentrate the bacteria by centrifugation at 10,000×g for 6 min.
3. Repeat the washing step twice more (steps 1 and 2). Resuspend in 30-60 μL of 20 mM HEPES buffer pH 7.7, depending on sizes of pellets.
4. Mix 10 μL of the resuspended cells from step 4 with 5 μL prepGEM™ protease (0.025 U) and 1 μL lysozyme (5 μg). Make up the final volume to 100 μL by adding 84 μL HEPES buffer.
5. Program a PCR cycling machine (or similar) as below:
   Incubate the mixture at 37° C. for 15 min.
   Incubate the mixture at 75° C. for 15 min.
   Incubate the mixture at 95° C. for 15 min.
6. Sediment the cell debris by centrifuging the extraction tube at 10,000×g for 5 min.
7. Use 5 μL of the DNA supernatant for PCR amplifications.

### 2. PCR Amplification

A Multiplex PCR employing eight primers was performed. Reagents used are listed in Table 10 below.

TABLE 10
PCR reagents

| PCR stock reagents (16.8 μL) | DNA template | MilliQ water |
|---|---|---|
| PCR buffer (10x) - 10.0 μL | 5 μL of | 28.2 μL |
| MgCl₂ (25 mM) - 4.0 μL | extracted DNA | |
| dNTPs (2 mM) - 2.5 μL | | |
| BSA - 0.5 μL | | |
| MF1F (10 μM) - 0.75 μL | | |
| MF1R (10 μM) - 0.75 μL | | |
| MF2F (10 μM) - 0.75 μL | | |
| MF2R (10 μM) - 0.75 μL | | |
| MF3F (10 μM) - 0.375 μL | | |
| MF3R (10 μM) - 0.375 μL | | |
| MF4F (10 μM) - 3 μL | | |
| MF4R (10 μM) - 3 μL | | |
| Ampli Taq DNA polymerase | | |
| (5 Unit/μL) - 0.3 μL | | |

Final volume of 50 μL in each PCR tube

In various experiments, a label such as a fluorescent tag was used for one primer in each primer pair. The primer was either FAM or HEX. Other possible primers are described above. Different labels were used for each labelled primer to allow the amplicons to be readily differentiated. Primers are discussed in detail above and below. F in primer

name denotes a forward primer. R in primer name denotes a reverse primer. It is apparent that the label can be on either the forward or the reverse primer.

A positive control (1 μL of E. coli K12 MG1655 DNA) and a negative control (all the reagents except the DNA template) were also carried out in parallel. The PCR amplification conditions used are shown below:

1. 94° C. for 3 min.

2. 94° C. for 30 s.

3. 53° C. for 30 s.

4. 72° C. for 15 s, cycle steps 2-4 for 25 cycles.

5. 72° C. for 10 min.

6. 22° C. holding until stored or analysed.

### 3. Amplicon Size Determination

1 μL of the PCR products and 1 μL internal standards prepared from E. coli to calibrate sizes of unknown amplicons) were loaded and mixed into one well of the plate for running capillary electrophoresis. Capillary electrophoresis was carried out using the 3100 Genetic Analyzer, using ROX 350 as size standard. Up to 96 wells/samples can be run simultaneously, taking 30 to 40 min.

The raw data output from 3100 Genetic Analyzer was analyzed using either the programs GeneScan® or GeneMapper® (Applied Biosystems USA).

Internal size marker amplicons made from the Escherichia coli 16S rRNA gene were run with the sample, to provide internal validation and correction of amplicon sizes.

### 4. Bacterial Identification

Having obtained the electropherogram (EPG) for the sample, the bacterial composition was identified based on the sizes determined from the position of the peaks along the time axis as compared with the size standards. Every pair of primers was designed from two conserved regions of bacterial 16S or 23S rRNA genes which flank a highly variable length region of the gene. The PCR products that are synthesized from different bacterial genomes will most likely (but not always) possess different sizes for at least one of four amplicons. Therefore, each peak can be assigned to its bacterial species based on size. This assignment was done by comparing the theoretical size(s) of amplicons calculated from bacterial sequences of 16S and 23S rRNA genes with the actual size(s) determined from the electropherograms and in some cases from predicted fragment sizes based on DNA sequences. Some slight variance in the actual value was observed due to the limits in the precision of the equipment. In these experiments, although internal standards provided by the manufacturer were used, further calibration was achieved by adding internal size standards generated from fluorescently labelled PCR products generated from regions within the 16S rRNA gene of E. coli. In this experiment the internal standards were a mixture of five fluorescently labelled PCR products amplified from E. coli K12 MG1655 genome although it should be understood that any sequences of appropriate length could be used. These five internal standards were designed to have sizes flanking each of the amplicons.

The first three amplicons (of the bacterial oligonucletide rRNA gene) span different size ranges without any known overlapping. The fourth amplicon(s) can overlap in size with the other amplicons and so are differentiated by using a different fluorescent label. Bacterial identification is performed by a process of elimination for each amplicon. By having four sets of primers, a quarto-checked identification process is carried out on each organism. This identification process can be automated by a computer program. The program is designed to identify the bacterium or bacteria present, using a combination of four amplicon sizes specific to each bacterium.

As is accepted in the art, unwanted amplicons in a PCR can be greatly reduced by the use of specific blocking agents such as Peptide Nucleic Acids (PNAs), or Locked Nucleic Acids (LNAs) or other such high affinity nucleic acid binding agents. In this way interfering amplicons such as those arising from the 16S-like sequences in the mitochondrial genome.

It should also be understood that additional primers can be added to the set where greater discrimination is required. For example, this enhancement may be required where only specific strains of a bacterium are implicated in clinical manifestations.

### 5. PCR Primers.

The sequences of the primers used herein are listed above.

6-carboxyfluorescin (FAM) is a fluorescent tag attached at the 5′ end of some primers. Hexachloro-6-carboxy-fluorescine (HEX) is a fluorescent tag attached at the 5′ end of some primers. Different tags can be used when amplicons can overlap, to differentiate between amplicons.

### 6. Database of Amplicons

A database of experimental amplicon sizes for pathogenic microflora may be generated by harvesting an isolated colony of each type strain of bacterium shown, processing the pure culture as described, and measuring the sizes of Amplicons I to IV. In parallel, 16S rRNA gene and partial 23S rRNA gene genes have been amplified from DNA extracted from the same cultures, and sequenced to provide verification of the sequences or to provide new data. The primer binding sites have been identified in the generated sequences, and the theoretical amplicon sizes estimated by sequence length including the primer positions. There is generally very good agreement between the two values—usually within 1%.

A computer program can be written to identify the detected bacteria from the database. To perform the task manually is relatively simple when the number of expected organisms is relatively small or only a single organism is present, but when a more diverse panel of bacteria are known to be potentially present, or when more than one species of organism is present in a sample then the computer program is of greater value. Multiple databases can be generated each focussing on a specific sample type.

### 7. Preparation of Fluorescent Internal-Standard Oligonucleotides for Calibrating Experimental Results.

To further calibrate the AB3100 column for sizing oligonucleotides, five reverse primers were designed, each to be used with primer MF2F to make internal-standard fluorescently-labeled oligonucleotides of known length. They were:

ME20R (SEQ ID NO: 31):
5′ ACA ACC CGA AGG CCT TCT 3′ (Product 90 bases)
ME21R (SEQ ID NO: 32):
5′ CGT CAA TGA GCA AAG GTA 3′ (Product 145 bases)
ME22R (SEQ ID NO: 33):
5′ CGC CGC TGC TGG CAC GGA 3′ (Product 190 bases)
ME23R (SEQ ID NO: 34):
5′ TGC GCT TTA CGC CCA GTA 3′ (Product 240 bases)
ME24R (SEQ ID NO: 35):
5′ GCT ACA CCT GGA ATT CTA 3′ (Product 350 bases)

However, it is understood that the generation of internal standards is not limited to these sequences and that any selection of primers and could be used provided the amplicons generated have utility in normalising estimated lengths in the required range.

### Results

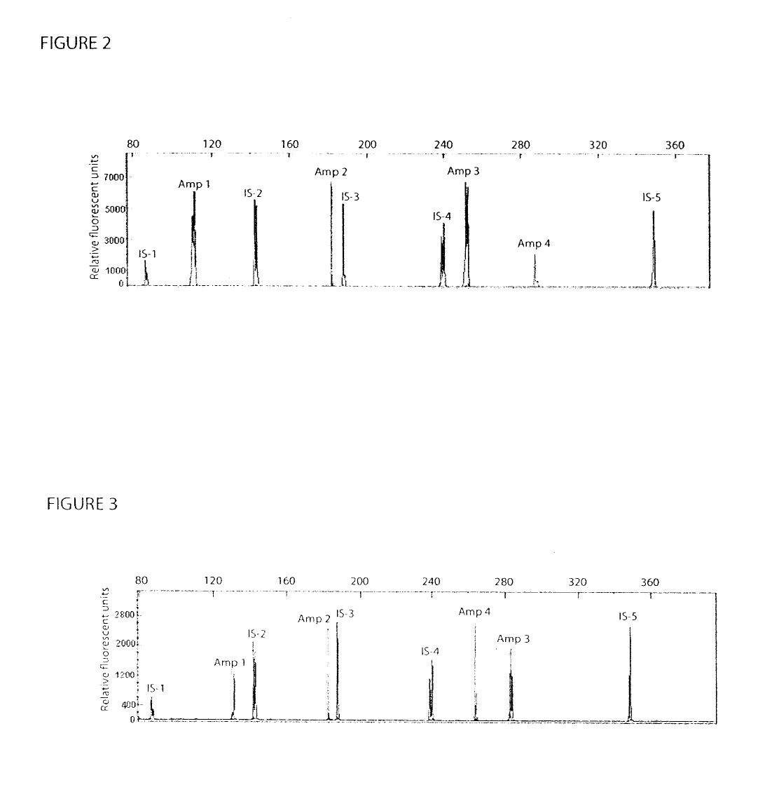

The profile obtained from an isolated colony of the bacterium Escherichia coli K12, strain MG1655 is shown in FIG. 2. Four amplicon peaks are shown, with sizes of 113.7, 184.1, 252.5, and 290.4 bases. Reference to the sequence database confirms that these amplicons correspond to those expected for E. coli (expected 113, 184, 250, and 292 bases, respectively).

### Example 2

### DNA Extraction from Bacterial Colonies (Lactobacillus gasseri)

This experiment describes the extraction of DNA from Lactobacillus gasseri type DSM 20243 colonies on agar plates and its use in the identification of the bacteria using the methods described herein. Reagents and Methods were as described in Example 1 above.

The profile obtained from an isolated colony of the bacterium Lactobacillus gasseri type DSM 20243 is shown in FIG. 3. Four amplicon peaks are shown, with sizes of 134.1, 185.2, 284.8, and 265.8 bases. Reference to the sequence database confirms that these amplicons correspond to those expected for Lactobacillus gasseri (expected 134, 184, 286, and 265 bases respectively).

### Example 3

### DNA Extraction from Bacterial Colonies (Staphylococcus aureus)

This experiment describes the extraction of DNA from Staphylococcus aureus ATCC 25923 colonies on agar plates and its use in the identification of the bacteria using the methods described herein. Reagents and Methods were as described in Example 1 above.

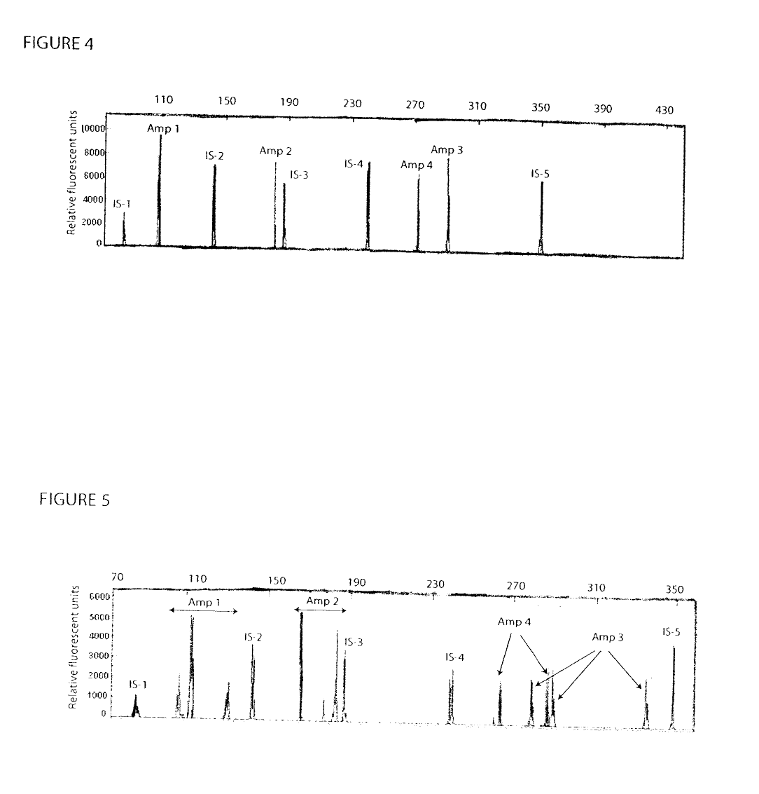

The profile obtained from an isolated colony of the bacterium Staphylococcus aureus ATCC 25923 is shown in FIG. 4. Four amplicon peaks are shown, with sizes of 111.3, 184.1, 291.8, and 273.1 bases. Reference to the sequence database confirms that these amplicons correspond to those expected for Staphylococcus aureus (expected 111, 184, 293, and 273 bases respectively).

### Example 4

### Identification of BV Microflora

This experiment describes the identification of microflora associated with human female genital tract infection broadly known as Bacterial Vaginosis (BV). When samples of bacteria to be analyzed are BV swab samples (or reasonably pure cultures of a pathogenic bacterium as described above) the speed of identification is approximately 7 hours, from start of analysis to identification. This is potentially useful clinically, because the physician would obtain the identity of an infectious bacterium within a working day. Advantageously, this method is able to identify non-culturable bacteria, which will give a unique set of Amplicon sizes enabling identification. Any bacterium representing approximately 5% or more within a mixed culture can be detected and identified if its profile is present in the database. It is understood that as the knowledge base around the infectious organisms associated with this disease expands, so will the database. Reagents were as described in Example I above.

### Methods

Suspend vaginal swab (or pipetted) sample in 1 mL of buffer. Extract DNA by following steps 2-7 of the method described in Example 1. Amplicon generation and analysis was performed as per Example 1.

### Results

The profile from the bacterial vaginosis positive swab exhibits 2 or 3 peaks for each of the four amplicons (FIG. 5). Amplicon I (109.1, 115.6 and 133.0 bases), amplicon II (169.0, 180.4, and 186.3 bases), amplicon III (279.9, 290.3 and 336.1 bases) and amplicon IV (265.6, and 288.4 bases). These peaks best describe a vaginal microflora with three bacteria present: (i) a Gardnerella species (G. vaginalis strain DSM 4944: expected 110, 167, 334 and 290 bases); (ii) a member of the Bacteroidetes family, (Prevotella. bivia DSM 20514 expected: 116, 179, 291 and 269), and (iii) and a bacterium related to Megasphaera elsdenii, (strain DSM 20460 expected: 116, 185, 281 and 290 bases). The 133.0 bases peak is inferred to be human 16S rRNA gene from human mitochondrial DNA, which gives expected peaks at 133 and 170 bases.

### Example 5

### Identification of Normal Vaginal Microflora

This experiment describes the identification of microflora associated with normal human female genital tract. Reagents were as described in Example 1 above. Methods were as described in Example 4 above.

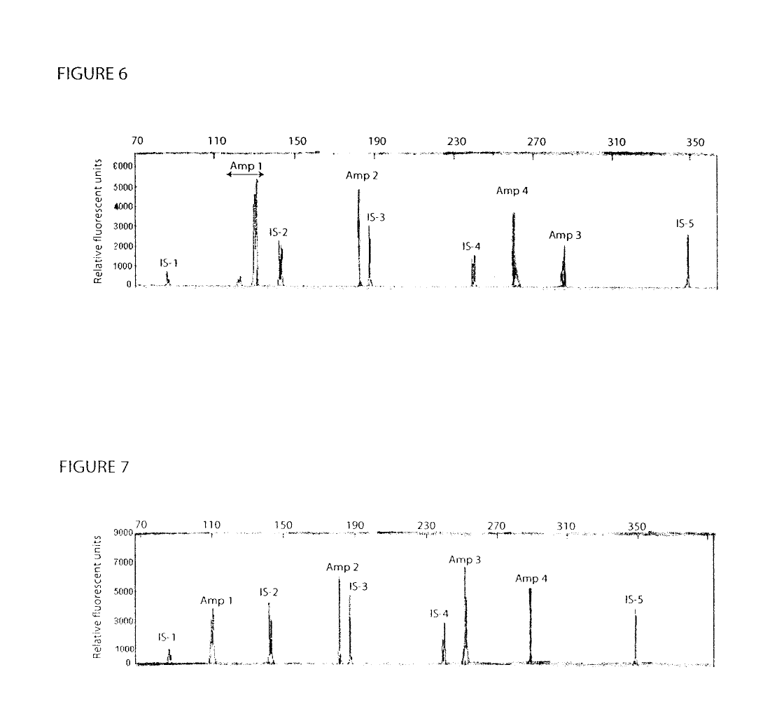

The profile obtained from a high vaginal swab, taken from a normal female subject not suffering from Bacterial Vaginosis, is shown as FIG. 6. Two peaks in amplicon 1 (124.8 bases and 132.8 bases), and a single peak for amplicons II-IV (185.6, 285.8, and 262.7 bases respectively), can be seen. These correspond to the expected peaks for the normal vaginal bacteria species Lactobacillus iners DSM 13335 (expected: 134, 184, 287, and 262 bases), and another Lactobacillus species Lactobacillus jensenii (expected: 126, 184, 287, and 262 bases). Although a similar pattern could be expected for other Lactobacillus species, the conclusion can be drawn that the normal microbial flora can be differentiated by this multiplex method.

### Example 6

### Identification of Microflora in an Infected Ear

This experiment describes the identification of microflora present on a swab from a patient with an infected ear. Reagents were as described in Example 1 above. Methods were as described in Example 4 above.

The profile generated from a swab obtained from an infected ear is shown in FIG. 7. The profile contains four amplicons, suggesting one predominant bacterium. The base lengths of the amplicons are 110.9, 184.2, 292.5, and 273.3 bases. This profile is in close agreement with the profile of the Staphylococcus aureus type strains and Staphylococcus epidermidis (S. epidermidis ATCC 12228 expected: 111, 184, 292 and 273 bases. As confirmation of the diagnosis, the hospital laboratory cultured Staphylococcus aureus from the sample.

### Example 7

### Identification of Microflora of Urine

This experiment describes the identification of microflora present in the urine of a patient with a bladder infection. Reagents were as described in Example 1 above. Methods were as described in Example 4 above, with bacteria obtained by sedimenting 0.3 ml of urine by centrifugation at 10000×g for 5 min.

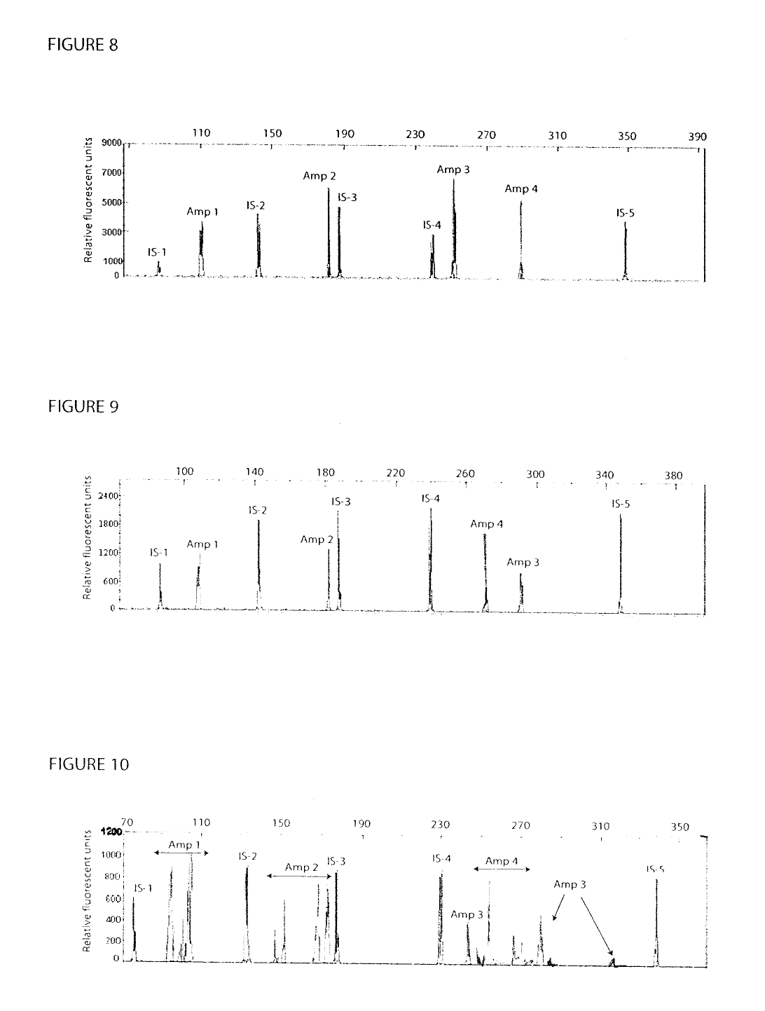

The profile generated from a swab obtained from the urine sample is shown in FIG. 8. The profile contains four amplicons, suggesting one predominant bacterium was present. The base lengths of the amplicons are 113.3, 184.0, 252.6 and 291.0 bases, which is in close agreement with the Escherichia coli strain described in Example 1. As confirmation of the diagnosis, the hospital laboratory identified Escherichia coli as the culturable bacterium in the sample.

### Example 8

### Identification of Microflora of Spine Biopsy

This experiment describes the identification of microflora present in spine biopsy. Reagents were as described in Example 1 above. Additional reagents were:

### Phosphate Buffer

100 ml of 0.1M phosphate buffer at pH8; 9.32 ml of 1M Na₂HPO₄ was mixed with 0.68 ml of 1M NaH₂PO₄ and 90 ml of milliQ water

### SDS Lysis Buffer

60 ml of SDS lysis buffer; 16 ml H2O was added to 6 g SDS (sodium dodecylsulphate). 30 ml of 1.0 M Tris (pH8 at 25° C.) was added followed by 1.2 ml of 5M NaCl. The solution was stirred until dissolved and made up to 60 ml by adding milliQ water.

### Lysozyme Bead Mix

Lysozyme was made to a concentration of 10 mg per ml. Beads were a mixture of 1.0 mm zirconium beads and 2.5 mm zirconium beads.

### Method.

1. The sample was immersed in 270 μl of phosphate buffer. 30 μl of lysozyme was added with beads and incubated for 1 hour.
2. 300 ul of SDS lysis buffer was added and 300 μl of chloroform:isoamyl alcohol mix (24:1).
3. Vials were shaken in a FastPrep machine (Bio101) at 4.0 m/s for 40 sec.
4. The supernatant was transferred to a new tube and centrifuged at 10000×g for 5 minutes and the supernatant transferred to a new tube (approximately 650 μl).
5. 7M NaOAc was added to give a final concentration of 2.5 M, vortexed, and centrifuged at 10000×g for 5 minutes.
6. The supernatant was transferred to a new tube and 0.54 volumes of isopropanol added. The solution was mixed and left to stand at room temperature for 15 min.
7. The tube was then centrifuged at 10000×g for 5 minutes, the supernatant decanted and discarded and the pellet washed twice in 70% ethanol.
8. The DNA pellet was dried and 1 then resuspended in 60 μl of water.
9. A 1:5 or 1:10 dilution of the DNA was carried for some of the samples and 5 μL used for a 25 μL PCR reaction.

Amplicon generation and analysis was performed as per Example 1. The profile generated from a swab obtained from the spine biopsy is shown in FIG. 9. The profile contains four amplicons, suggesting one predominant bacterium. The base lengths of the amplicons are 111.0, 184.3, 292.5 and 273.3 bases, which is in close agreement with the profile of a Staphylococcus species described in example 6. As confirmation of the diagnosis, the hospital laboratory cultured Staphylococcus epidermidis from the sample.

### Example 9

### Identification of Microflora of Sputum

This experiment describes the identification of microflora present in a sputum sample provided by a patient with cystic fibrosis. Reagents were as described in Example I above. Methods were as described in Example 4 above, with bacteria obtained by resuspending the sputum sample in buffer and then by centrifugation at 10000×g for 5 min.

The profile generated from sputum sample is shown in FIG. 10. The profile contains many peaks within each amplicon range, likely containing at least four different bacteria at a detectible level. A few of the predominant peaks were consistent with profiles that had been seen in pure cultures. There was a set of amplicon peaks at 116.1, 179.7, 291.8, and 265.9 bases, consistent with a prominent Bacteroidetes family member. Another set of peaks at 111.6, 185.3, 287.3, and 262.0 bases is consistent with a prominent Streptococcus species (Streptococcus pyogenes DSM 20565 type strain gives amplicons 110.8, 186.1, 286.7 and 262.4 bases (observed)). A third set of peaks at 109.2, 184.9, 255.9 and 279.7 bases is consistent with a Burkholderia species (B. cepecia ATCC 25416 gives amplicons 107.7, 185.4, 255.0 and 278.6 bases). Further peaks at 118.2 bases (amplicon 1), 159.6 and 164.7 bases (amplicon 2s), 295.0 and 327.4 bases (amplicon 3s), and 268.9 and 280.5 bases (amplicon 4s) could not be assigned from the database of bacterial amplicon profiles available at the time. The hospital laboratory characterized the sample as containing “mixed oral flora” without specifying the many types of bacteria present.

### Discussion of Examples

As every pair of primers was designed from two conserved regions of bacterial 16S or 23S rRNA genes which flank a highly variable length region of the gene, the PCR products that are synthesized from different bacterial genomes will most likely (but not always) possess different sizes for at least one of four amplicons. Therefore, each peak can be assigned to its bacterial species based on size. This assignment was done by comparing the theoretical size(s) of amplicons calculated from bacterial sequences of 16S and 23S rRNA genes with the actual size(s) determined from the electropherograms and in some cases from predicted fragment sizes based on DNA sequences. Some slight variance in the actual value was observed due to the limits in the precision of the equipment. In these experiments, although internal standards provided by the manufacturer were used further standardization was achieved by adding internal standards generated from fluorescently labelled PCR products generated from regions within the 16S rRNA gene of E. coli. In this experiment the internal standards were a mixture of five fluorescently labeled PCR products amplified from E. coli K12 MG1655 genome although it is understood that any sequences of appropriate length could be used. These five internal standards were designed to have sizes flanking each of the amplicons.

As is also accepted in the art, additional primers can be added to the set where greater discrimination is required. For example, this may be required where only specific strains of a bacterium are implicated in clinical manifestations.

It will be appreciated that it is not intended to limit the invention to the above examples only, many variations, which may readily occur to a person skilled in the art, being possible without departing from the scope thereof as defined in the accompanying claims.

### INDUSTRIAL APPLICATION

Methods for detecting and identifying known and unknown bioagents, particularly bacteria, by nucleic acid amplification and amplicon size determination are described herein. Primers, kits, and systems for use in the identification of bioagents are also provided. The need for identification of microflora in industry is commonplace. Examples are in the food processing industry and also in waste treatment. The latter is particularly relevant as it is a microbial process.

1. A method for the identification of a bioagent present in a sample, the method comprising:
(a) contacting nucleic acid present in said sample with:
(i) a first pair of primers designed to produce a first amplicon from a 16S rRNA gene or a 23S rRNA gene under amplification conditions;
(ii) a second pair of primers designed to produce a second amplicon from a 16S rRNA gene or a 23S rRNA gene under amplification conditions;
(iii) a third pair of primers designed to produce a third amplicon from a 16S rRNA gene or a 23S rRNA gene under amplification conditions; and
(b) amplifying said nucleic acid with said first, second and third pair of primers;
(c) determining the length of the amplicons produced; and
(d) identifying the bioagent on the basis of the length of the amplicons.

2. A method as claimed in claim 1 further comprising contacting the nucleic acid present in said sample with a fourth, fifth or sixth pair of primers designed to produce a fourth, fifth or sixth amplicon from a 16S rRNA gene or 23S rRNA gene under amplification conditions.

3. A method as claimed in claim 2 wherein the first, second, third, fourth, fifth or sixth pair of primers is designed to selectively amplify a region of a 16S rRNA gene.

4. A method as claimed in claim 3 wherein the region of the 16S rRNA gene comprises nucleotides 8 to 121 or nucleotides 8 to 120 or a region between nucleotides 7 and 122.

5. A method as claimed in claim 3 wherein the first, second, third, fourth, fifth or sixth pair of primers consists of a first primer that hybridizes to the 16S rRNA gene in the region of nucleotides 8 to 121, nucleotides 8 to 68 or nucleotides 8 to 43, and a second primer that hybridizes to the 16S rRNA gene in the region of nucleotides 8 to 121, nucleotides 98 to 121 or nucleotides 104 to 121.

6. A method as claimed in claim 3 wherein the first, second, third, fourth, fifth or sixth pair of primers consists of a first primer of SEQ ID No. 1 or 2 and a second primer selected from SEQ ID NO.s 3 to 11.

7. A method as claimed in claim 3 wherein the first, second, third, fourth, fifth or sixth pair of primers consists of a first primer comprising 15 or more contiguous nucleotides of SEQ ID No. 2 and a second primer comprising 15 or more contiguous nucleotides of SEQ ID No. 11.

8. A method as claimed in claim 3 wherein the first, second, third, fourth, fifth or sixth pair of primers is designed to selectively amplify a region of a 16S rRNA gene comprising nucleotides 310 to 588, nucleotides 310 to 523, nucleotides 310 to 537, nucleotides 338 to 523, nucleotides 338 to 537, nucleotides 338 to 588, nucleotides 340 to 523, nucleotides 340 to 537 or nucleotides 340 to 588, or a region between nucleotides 309 and 589.

9. A method as claimed in claim 3 wherein the first, second, third, fourth, fifth or sixth pair of primers consists of a first primer that hybridizes to the 16S rRNA gene in the region of nucleotides 310 to 588, nucleotides 310 to 368 or nucleotides 338 to 365, and a second primer that hybridizes to the 16S rRNA gene in the region of nucleotides 310 to 588, nucleotides 504 to 588 or nucleotides 504 to 537.

10. A method as claimed in claim 3 wherein the first, second, third, fourth, fifth or sixth pair of primers consists of a first primer of SEQ ID No. 12 or 13 and a second primer of SEQ ID No. 14 or 15.

11. A method as claimed in claim 3 wherein the first, second, third, fourth, fifth or sixth pair of primers consists of a first primer comprising 15 or more contiguous nucleotides of SEQ ID No. 13 and a second primer comprising 15 or more contiguous nucleotides of SEQ ID No.

12. A method as claimed in claim 2 wherein the first, second, third, fourth, fifth or sixth pair of primers is designed to selectively amplify a region of a 23S rRNA gene.

13. A method as claimed in claim 12 wherein the region of the 23S rRNA gene comprises nucleotides 232 to 517, nucleotides 232 to 481, nucleotides 232 to 485, nucleotides 241 to 517, nucleotides 241 to 481, or nucleotides 241 to 485, or a region between nucleotides 231 and 518.

14. A method as claimed in claim 12 wherein the first, second, third, fourth, fifth or sixth pair of primers consists of a first primer that hybridizes to the 23S rRNA gene in the region of nucleotides 232 to 517 or nucleotides 232 to 256, and a second primer that hybridizes to the 23S rRNA gene in the region of nucleotides 232 to 517, nucleotides 442 to 517, nucleotides 442 to 485 or nucleotides 459 to 485.

15. A method as claimed in claim 12 wherein the first, second, third, fourth, fifth or sixth pair of primers consists of a first primer of SEQ ID No. 16, 17 or 18 and a second primer of SEQ ID No. 19 or 20.

16. A method as claimed in claim 12 wherein the first, second, third, fourth, fifth or sixth pair of primers consists of a first primer comprising 15 or more contiguous nucleotides of SEQ ID No. 18 and a second primer comprising 15 or more contiguous nucleotides of SEQ ID No. 20. See Tables 5 and 6 below for SEQ ID Nos. 16 to 20.

17. A method as claimed in claim 12 wherein the first, second, third, fourth, fifth or sixth pair of primers is designed to selectively amplify a region of a 23S rRNA gene comprising nucleotides 1654 to 1971, nucleotides 1654 to 1945, nucleotides 1654 to 1854, nucleotides 1654 to 1843, 1656 to 1971, nucleotides 1656 to 1945, nucleotides 1656 to 1854, nucleotides 1656 to 1843, 1661 to 1971, nucleotides 1661 to 1945, nucleotides 1661 to 1854, nucleotides 1661 to 1843, or a region between nucleotides 1653 and 1972.

18. A method as claimed in claim 12 wherein the first, second, third, fourth, fifth or sixth pair of primers consists of a first primer that hybridizes to the 23S rRNA gene in the region of nucleotides 1654 to 1854, nucleotides 1654 to 1704 or nucleotides 1654 to 1677, and a second primer that hybridizes to the 23S rRNA gene in the region of nucleotides 1654 to 1854, nucleotides 1818 to 1854 or nucleotides 1889 to 1971.

19. A method as claimed in claim 12 wherein the first, second, third, fourth, fifth or sixth pair of primers consists of a first primer of SEQ ID No. 21, 22 or 23 and a second primer of SEQ ID No. 24, 25, 26, 27 or 28.

20. A method as claimed in claim 1 wherein the method further comprises:
a) contacting a nucleic acid of known identity with one or more primer pairs adapted to amplify said nucleic acid of known identity under amplification conditions to produce one or more calibration amplicons of a known length;
b) amplifying said nucleic acid with said one or more primer pairs; and
c) determining the length of the amplicons produced.

21. A method as claimed in claim 20 wherein the one or more primer pairs comprise a first primer and a second primer, wherein the first primer is 5′ TCC TAC GGG AGG CAG CAG 3′ and the second primer is chosen from the group consisting of 5′ ACA ACC CGA AGG CCT TCT 3′,5′ CGT CAA TGA GCA AAG GTA 3′,5′ CGC CGC TGC TGG CAC GGA 3′,5′ TGC GCT TTA CGC CCA GTA 3′, and 5′ GCT ACA CCT GGA ATT CTA 3′.

22. A method as claimed in claim 1 wherein the bioagent is a bacterium.

23. A method as claimed in claim 1 wherein the amplification occurs via PCR.

24. A method as claimed in claim 1 wherein the length of the amplicons is determined by electrophoretic separation.

25. A method as claimed in claim 1 wherein at least one primer of each primer pair is labelled to allow for visual detection of amplicons.

26. A method as claimed in claim 25 wherein at least one primer of each primer pair is labelled with one of FAM, HEX, JOE, ROX, TMARA, TET.

27. A method as claimed in claim 26 wherein each labelled primer has a different label to facilitate identification of amplicons.
